(19) United States
(12) Patent Application Publication
(10) Pub. No.: US 2022/0411787 A1
(43) Pub. Date: Dec. 29, 2022

(54) GENETICALLY-DIRECTED SPARSE AND COMPLETE LABELING OF BRAIN CELLS

(71) Applicant: THE REGENTS OF THE UNIVERSITY OF CALIFORNIA, OAKLAND, CA (US)

(72) Inventors: Xiangdong William Yang, LOS ANGELES, CA (US); Matthew B. Veldman, LOS ANGELES, CA (US)

(73) Assignee: THE REGENTS OF THE UNIVERSITY OF CALIFORNIA, OAKLAND, CA (US)

(21) Appl. No.: 17/756,336

(22) PCT Filed: Nov. 23, 2020
(86) PCT No.: PCT/US20/61852

§ 371 Date: May 23, 2022

Related U.S. Application Data
(60) Provisional application No. 62/939,599, filed on Nov. 23, 2019.

(51) Int. Cl. C12N 15/10; C12N 15/90

(52) U.S. Cl. CPC C12N 15/1065; C12N 15/907; C12N 2800/30

(57) ABSTRACT

Constructs and methods are described for producing sparse and stochastic labeling of cells expressing one or more site-specific recombinases in a host mammal. Described is a nucleic acid construct comprising, in operable linkage, a translation start site, an optional spacer, a polycytosine mononucleotide repeat, and an open reading frame (ORF), wherein the polycytosine repeat and the ORF are out of frame with respect to the translation start site. The simple, general, and scalable solution for genetically-directed sparse cell labeling allows the visualization of the complete cellular morphology of cells. Representative examples of cells to be visualized using sparse labeling include, but are not limited to, neurons or non-neuronal cells in the central nervous system (including brain), peripheral nervous systems, and other peripheral tissues.

This application claims benefit of U.S. provisional patent application No. 62/939,599, filed Nov. 23, 2019, the entire contents of which are incorporated by reference into this application.

### ACKNOWLEDGEMENT OF GOVERNMENT SUPPORT

This invention was made with government support under Grant Number MH106008, awarded by the National Institutes of Health. The government has certain rights in the invention.

### BACKGROUND

The mammalian brain consists of an astronomical number of neurons (e.g. an average of about 86 billion in the human brain and 100 million in the mouse brain (Herculano-Houzel, 2012; Herculano-Houzel et al., 2006)). Each neuron has its complex shape with dendrites, axons, and pre- and post-synaptic structures. Together, the shape of an individual neuron determines its connectivity and confers and constrains its function in a neural circuit. The ability to evaluate the full morphology of individual neurons brainwide and to do so at a simple, reproducible, and scalable manner could help address many important questions in neurobiology, such as neuronal cell type classification (Zeng and Sanes, 2017), connectome of neural circuits (Swanson and Lichtman, 2016), neuronal plasticity (Holtmaat and Svoboda, 2009), and neuropathology (Duman and Aghajanian, 2012; Adalbert and Coleman, 2013).

Neurons are densely packed in the brain, and even those with similar molecular or genetic markers often have overlapping dendritic and axonal processes, which preclude the ready visualization of their morphology. Thus, to unveil the detail morphology of a neuron, it often requires a sparse labeling method that randomly labels a small subset of neurons, highlighting their dendritic and axonal processes. The first such tool was the classical Golgi staining, which allows sparse and stochastic labeling of the neurons in the brain to reveal their complete morphology (Swanson and Lichtman, 2016). However, the silver staining reaction (i.e. Golgi staining) is time-consuming and not compatible with modern techniques to selectively study genetically-defined neurons or co-detection of other mRNA or protein markers.

Several methods have been developed to achieve sparse labeling of genetically defined single neurons (Luo et al, 2008; Jefferis and Livet, 2012). In vitro dye filling of genetically-labeled neurons by microinjection can give fine morphological details and be coupled with electrophysiological recording and single cell RNA-seq technologies (e.g. Gouwens et al, 2019). However, such a method is labor intensive and poorly scalable, and the analyses are limited to the partial morphology of a neuron within a brain slice. Another popular method is sparse labeling of neurons using diluted viral vectors (e.g. AAV, rabies or pseudorabies, and lentivirus) to deliver various reporters (e.g. Economo et al., 2016; Chan et al., 2017). Such methods are somewhat limiting due to the invasiveness, non-random distribution and tropism of the viruses, inability to study embryonic or early postnatal neurons, and neurotoxicity associated with long-term viral infection or high-level reporter expression (Nassi et al., 2015).

A few Cre-dependent mouse genetic tools have been used for sparse cell labeling (Luo et al., 2008; Jefferis and Livet, 2012). One such method is combining an inducible Cre-ER recombinase with low-dose tamoxifen induction, resulting in exquisite labeling of a subset of Cre+ neurons and their processes (Badea et al, 2003; Badea et al., 2009; Wang et al., 2019). However, such an approach has the limitation of some background recombination in uninduced mice, difficulty to control the labeling frequency, and the limited number of available Cre-ER lines and the inability to use the large collection of existing conventional Cre mouse lines (Hayashi and McMahon, 2002; Hameyer et al., 2007; Wang et al., 2019). The Brainbow method of Cre-dependent expression of a stochastic combination of fluorescent proteins leads to multicolor labeling of neuronal cell bodies, dendrites and axons (Livet et al., 2007). However, the labeling frequency of these mice is often too dense to readily discern the full dendritic and axonal morphology of individual labeled neurons.

A third elegant genetic method to enable Cre-dependent sparse and stochastic cell labeling is called Mosaic Analysis with Double Markers (MADM) (Zong et al., 2006). This method uses two knockin mouse lines that have targeted insertion in the homologous genomic loci two spilt halves of XFPs of distinct colors separated by LoxP sites. The cells in MADM mice are non-fluorescent at the baseline, but upon Cre-mediated chromosomal recombination during mitosis, the two daughter cells reconstitute the expression of two distinctly colored XFPs. The advantage of MADM, similar to MARCM in Drosophila, is the possibility of creating two daughter cell clones expressing different XFPs and possible carrying either wildtype or homozygous mutations (Lee and Luo, 1999). Some limitations of the MADM method include the need to generate at least triple transgenic mice for each experiment, and the requirement to mouse lines expressing Cre during mitosis and therefore it cannot be used with many post-mitotically expressed, neuronal or non-neuronal cell type specific Cre lines.

There remains a need for complete yet non-toxic, sparse labeling of neurons and non-neuronal cells (i.e. astrocytes, oligodendrocytes, microglia and blood vessel cells) in the brain. There remains a need for simple and scalable solutions for both mesoscale and nanoscale studies of the detailed morphologies of defined cell types in the brain.

### SUMMARY

The materials and methods described herein provide a simple, general and scalable solution in a mammalian species for genetically-directed sparse cell labeling that allows the visualization of the complete cellular morphology of cells. Representative examples of cells to be visualized using sparse labeling include, but are not limited to, neurons or non-neuronal cells in the central nervous system (including brain), peripheral nervous systems, and other peripheral tissues. In one embodiment, described herein is a nucleic acid construct comprising, in operable linkage, a translation start site, an optional spacer, a polycytosine or polyguanine mononucleotide repeat, and an open reading frame (ORF), wherein the polycytosine or polyguanine repeat and the ORF are out of frame with respect to the translation start site.

In some embodiments, the mononucleotide repeat is a polycytosine repeat. In some embodiments, the polycytosine mononucleotide repeat consists of a minimum of 5 cytosines. In some embodiments, the polycytosine mononucleotide repeat consists of 5 to 25 cytosines. In some embodiments, the polycytosine mononucleotide repeat consists of 5 to 50 cytosines. In some embodiments, the polycytosine mononucleotide repeat consists of 22 cytosines (C₂₂). In some embodiments, the spacer comprises at least 3 base pairs, but it does not contain a translational STOP codon (i.e. TAG, TGA or TAA). In some embodiments, the spacer comprises between 3 and 1000 base pairs, but it does not contain a translational STOP codon (i.e. TAG, TGA or TAA). In some embodiments, the spacer comprises at least 3 and can be over 1000 base pairs, but it does not contain a translational STOP codon (i.e. TAG, TGA or TAA). In some embodiments, the spacer comprises between 30 and 100 base pairs. In some embodiments, the optional spacer sequence encodes one or two Myc tags.

In some embodiments, the ORF encodes a fluorescent protein. In some embodiments, the fluorescent protein is GFP, RFP, tdTomato, and/or mNeonGreen. In some embodiments, the ORF encodes an immunoreporter (e.g. an immunoreactive epitope tag-containing protein). In some embodiments, the ORF encodes a spaghetti monster immunoreporter with up to 10 epitope-tags inserted into a superfold GFP scaffold. In some embodiments, the immunoreporter comprises simian virus 5-derived epitope (V5), myelocytomatosis viral oncogene (Myc), hemagglutinin (HA), a FLAG tag, and/or OLLAS (Escherichia coli OmpF linker and mouse langerin). In some embodiments, the immunoreporter is a spaghetti monster immunoreporter with V5 epitope tags. Spaghetti monster fluorescent proteins are discussed in Viswanathan et al., 2015, Nature Methods 12(6): 568.

In some embodiments, the ORF encodes a functional protein that can activate or suppress gene expression, such as site-specific recombinase (e.g. Cre, Flp or FlpO, Dre, or Vika) or site-specific integrase (e.g. PhiC31, Bxb1). In some embodiments, the ORF encodes a genetically-encoded calcium indicator or “GECI” (e.g. GCaMP3, GCaMP5, GCaMP6, jGCaMP7, or R-GRECO1). In some embodiments, the ORF encodes one or more DNA or RNA programmable nucleases that enable genomic DNA or RNA editing (e.g. SpCas9, SaCas9 Cpf1, Cas13, ZFNs, TALENs).

In some embodiments, the ORF encodes a tandem fusion of two spaghetti monster immunoreporters with each immunoreporter consisting of up to 10 epitope tags inserted into the superfold GFP scaffold. In some embodiments, the ORF encodes a tandem fusion of two spaghetti monster immunoreporters with a total of up to 20 V5 epitope tags. In some embodiments, the ORF encodes a tandem fusion of two immunoreporters with a total of up to 20 epitope tags, which may include, but is not limited to, any combination of Myc tags, HA tags, FLAG tag, V5 tags, and OLLAS tag.

In some embodiments, the ORF encodes a membrane insertion signal. In some embodiments, the ORF is fused at the C-terminal with a farnesylation signal. In some embodiments, the farnesylation signal is a Ras CAAX domain.

In some embodiments, the ORF encodes a polypeptide or protein that has enzymatic activity. Examples of such polypeptides and proteins having enzymatic activity include those described in the following references: APEX labeling and BioID: Rhee, H. W. et al. Proteomic mapping of mitochondria in living cells via spatially restricted enzymatic tagging. Science 339, 1328-1331 (2013); Lam, S. S. et al. Directed evolution of APEX2 for electron microscopy and proximity labeling. Nat. Methods 12, 51-54 (2015); Li P., Li J., Wang L. & Di L. J. Proximity labeling of interacting proteins: application of BioID as a discovery tool. Proteomics 17, 10.1002/pmic.201700002 (2017); Roux, K. J., Kim, D. I., Raida, M. & Burke, B. A promiscuous biotin ligase fusion protein identifies proximal and interacting proteins in mammalian cells. J. Cell Biol. 196, 801-810 (2012).

In some embodiments, the construct further comprises a polyadenylation signal downstream from the ORF. In some embodiments, the construct further comprises a protein coding sequence between the translation start site (ATG) and the polycytosine mononucleotide repeat. In some embodiments, the construct further comprises a protein coding sequence inserted between the polycytosine mononucleotide repeat and the ORF. In some embodiments, the protein coding sequence is a cDNA or a genomic DNA. In some embodiments, the construct is that illustrated in FIG. 1.

In some embodiments, the construct further comprises a promoter, a transcriptional stop sequence, and two site-specific recombinase binding sites flanking the transcriptional stop sequence, wherein the promoter is upstream of the recombinase binding sites, and wherein each of the preceding elements is upstream of the translation start site. In some embodiments, the promoter is a cytomegalovirus early enhancer element and/or a chicken beta actin (CAG) promoter. In some embodiments, the transcriptional stop sequence contains at least one polyadenylation signal. In some embodiments, the recombinase binding sites are LoxP sites, and the LoxP sites are oriented such that Cre recombinase excises the transcriptional stop sequence. In some embodiments, the recombinase binding sites are Frt sites, and the Frt sites are oriented such that Flp recombinase excises the transcriptional stop sequence. In some embodiments, the recombinase binding sites are Vox sites, and the Vox sites are oriented such that Vika recombinase excises the transcriptional stop sequence. In some embodiments, the recombinase binding sites are Rox sites, and the Rox sites are oriented such that Dre recombinase excises the transcriptional stop sequence. In some embodiments, the recombinase binding sites are Vox sites, and the Vox sites are oriented such that Vika recombinase excises the transcriptional stop sequence. In some embodiments, the recombinase binding sites comprise one attB site and one attP site, which are specific for PhiC31 integrase. The attB and attP sites are oriented such that PhiC31 integrase excises the transcriptional stop sequence. In some embodiments, the recombinase binding sites comprise one attB site and one attP site, which are specific for BxB1 integrase. The attB and attP sites are oriented such that Bxb1 integrase excises the transcriptional stop sequence.

Also described is a nucleic acid construct comprising, in operable linkage, a translation start site, a spacer, a polyguanine mononucleotide repeat, an obligatory spacer of at least 3 base pairs, and an open reading frame (ORF), wherein the polyguanine mononucleotide repeat and the ORF are out of frame with respect to the translation start site. In some embodiments, the polyguanine mononucleotide repeat consists of at least 5 guanines. In some embodiments, the polyguanine mononucleotide repeat consists of between 5 and 25 guanines. In some embodiments, the polyguanine mononucleotide repeat consists of between 5 and 50 guanines. In some embodiments, the polyguanine mononucleotide repeat consists of 22 guanines (Ga). In some embodiments, the spacer comprises between 3 and 1000 base pairs. In some embodiments, the spacer comprises at least 30 base pairs. In some embodiments, the spacer sequence encodes one or two Myc tags.

In some embodiments, the ORF encodes a fluorescent protein. In some embodiments, the fluorescent protein is GFP, RFP, tdTomato, and/or mNeonGreen. In some embodiments, the ORF encodes an immunoreporter, such as, for example, an immunoreactive epitope tag-containing protein. In some embodiments, the ORF encodes a spaghetti monster immunoreporter with up to 10 epitope-tags inserted into a superfold GFP scaffold. In some embodiments, the immunoreporter comprises simian virus 5-derived epitope (V5), myelocytomatosis viral oncogene (Myc), hemagglutinin (HA), FLAG tag, and or OLLAS (Escherichia coli OmpF linker and mouse langerin). In some embodiments, the immunoreporter is a spaghetti monster immunoreporter with V5 epitope tags.

In some embodiments, the ORF encodes a tandem fusion of two spaghetti monster immunoreporters with each immunoreporter consisting of up to 10 epitope tags inserted into the superfold GFP scaffold. In some embodiments, the ORF encodes a tandem fusion of two spaghetti monster immunoreporters with a total of up to 20 V5 epitope tags. In other embodiments, the ORF encodes a tandem fusion of two immunoreporters with a total of up to 20 epitope tags, which maybe include but not limited to any combination of Myc tags, HA tags, FLAG tag, V5 tags, and or OLLAS (Escherichia coli OmpF linker and mouse langerin).

In some embodiments, the ORF encodes a membrane insertion signal. In some embodiments, the ORF is fused at the C-terminal with a farnesylation signal. In some embodiments, the farnesylation signal is a Ras CAAX domain. In some embodiments, the ORF encodes a polypeptide or protein that has enzymatic activity.

In some embodiments, the construct further comprises at least one polyadenylation signal downstream from the ORF.

In some embodiments, the DNA construct has two open reading frames in the following configurations. First, a promoter preceding the Open Reading Frame 1 (ORF1), which is followed by a mononucleotide repeat, which in turn is followed by an Open Reading Frame 2 (ORF2). In some embodiments, the mononucleotide repeats comprise polyguanine with 5 to 50 guanine nucleotide residues or polycytosine with 5 to 50 cytosine nucleotide residues. The number of nucleotides is selected so that ORF2 is not in the same open reading frame as ORF1 without a frameshift of the mononucleotide repeat.

In some embodiments, the ORF1 in the place of spacer is a cDNA or a genomic DNA. In some embodiments, this genomic DNA is an endogenous genomic DNA that encodes a protein. In some embodiments, a promoter is placed in front of the ORF1.

In some embodiments, the promoter is a cytomegalovirus early enhancer element and chicken beta actin (CAG) promoter.

In some embodiments, a translational stop codon flanked by two recombinase binding sites is placed between the ORF1 and the mononucleotide repeat, which is followed by a second open reading frame (i.e. ORF2). In some embodiments, the translational stop sequence is followed by a spacer sequence of 3 nucleotides or more, and the translation stop plus spacer cassette is flanked by two recombinase binding sites. The number of nucleotides is selected so that the ORF1 is not in the same open reading frame as in ORF1 without a frameshift of the mononucleotide repeat. Moreover, the space sequence as well as the recombinase binding sites are designed so that there will be no translational stop codon between ORF1 and ORF2 after at least one of the frameshifts of the mononucleotide repeat.

In some embodiments, the recombinase binding sites are LoxP sites, and the LoxP sites are oriented such that Cre recombinase excises the translational stop sequence. In some embodiments, the recombinase binding sites are Frt sites, and the Frt sites are oriented such that Flp recombinase excises the translational stop sequence. In some embodiments, the recombinase binding sites are Vox sites, and the Vox sites are oriented such that Vika recombinase excises the translational stop sequence. In some embodiments, the recombinase binding sites are Rox sites, and the Rox sites are oriented such that Dre recombinase excises the translational stop sequence. In some embodiments, the recombinase binding sites are Vox sites, and the Vox sites are oriented such that Vika recombinase excises the translational stop sequence. In some embodiments, the recombinase binding sites are consisted of one attB site and one attP site, which are specific for PhiC31 integrase. The attB and attP sites are oriented such that PhiC31 integrase excises the translational stop sequence. In some embodiments, the recombinase binding sites are consisted of one attB site and one attP site, which are specific for BxB1 integrase. The attB and attP sites are oriented such that Bxb1 integrase excises the translational stop sequence. In all cases, the recombinase mediated excision of the translational stop sites do not introduce additional translational stop sequence between the ORF and the mononucleotide repeats.

In some embodiments, the ORF2 encodes a fluorescent protein. In some embodiments, the fluorescent protein is GFP, RFP, tdTomato, and/or mNeonGreen. In some embodiments, the ORF2 encodes an immunoreporter, such as, for example, an immunoreactive epitope tag-containing protein. In some embodiments, the ORF2 encodes a spaghetti monster immunoreporter with up to 10 epitope-tags inserted into a superfold GFP scaffold. In some embodiments, the immunoreporter comprises simian virus 5-derived epitope (V5), myelocytomatosis viral oncogene (Myc), hemagglutinin (HA), FLAG tag and or OLLAS (Escherichia coli OmpF linker and mouse langerin). In some embodiments, the immunoreporter is a spaghetti monster immunoreporter with V5 epitope tags.

In some embodiments, the ORF2 encodes a tandem fusion of two spaghetti monster immunoreporters with each immunoreporter consisting of up to 10 epitope tags inserted into the superfold GFP scaffold. In some embodiments, the ORF2 encodes a tandem fusion of two spaghetti monster immunoreporters with a total of up to 20 V5 epitope tags. In other embodiments, the ORF2 encodes a tandem fusion of two immunoreporters with a total of up to 20 epitope tags, which maybe include but not limited to any combination of Myc tags, HA tags, FLAG tag, V5 tags, and or OLLAS (Escherichia coli OmpF linker and mouse langerin).

Also provided is a cell comprising a construct as described herein. In some embodiments, the cell is a vertebrate cell. In some embodiments, the cell is a mammalian cell. Examples of mammalian cells include, but are not limited to, a murine cell. Additionally provided is an animal comprising a cell as described herein. In some embodiments, the animal is murine (rodents, such as mice, rats), avian (chicken, turkey, fowl), bovine (beef, cow, cattle), ovine (lamb, sheep, goats), porcine (pig, swine), piscine (fish), non-human primates (e.g. marmosets), or other vertebrates (e.g. frogs, fishes). In a preferred embodiment, the animal is a rodent, such as a mouse or a rat.

Described herein is a method of producing sparse and stochastic labeling of Cre-expressing cells in a host mammal, the method comprising generating a mouse that expresses the construct as described herein. In some embodiments, the labeling reveals the complete morphology of the cells. For neurons and other specialized cell types, the complete morphology can include processes that extend from the cell, such as axons and dendrites. The method employs optimized constructs that overcome limitations of prior attempts at developing sparse and stochastic labeling of cells, allowing for visualization of full neuronal morphology from dendrites and axons to spines. The exemplary mouse cell types described herein utilize membrane-bound, direct fluorescent reporters and immune-reporters to illuminate morphologies of genetically-defined neurons. This provides a scalable platform to capture the three-dimensional morphologies of individual neurons for extensive analyses.

By introducing a stochastic translational switch (polycytosine or polyguanine repeats), one can achieve sparse expression of a desired gene. This technique can be applied to transgenes (i.e. exogenously inserted genes into the genome) as well as to endogenous genes. In the case of endogenous genes, the translation switch (polycytosine or polyguanine repeat) can be used to sparsely and stochastically switch on the expression of a fluorescent, immunoreporter or enzymatically active protein as a fusion protein to an endogenously expressed protein in a cell.

### BRIEF DESCRIPTION OF THE DRAWINGS

FIG. 1 is a schematic illustration of constructs as described herein. (A). Construct #1 has an endogenous genomic locus with a promoter preceding a transcriptional STOP sequence flanked by two recombinase binding sites (RBS1 and RBS2). Recombinase binding sites can be LoxP for Cre recombinase; Frt for Flp recombinase; Rox for Dre recombinase; Vox for Vika recombinase; attB and attP for PhiC31 integrase; or attB and attP for Bxb1 integrase. The promoter can be any transcriptional promoter (e.g. CAG promoter or an inducible tTA driven TetO promoter). Spacer is 3 to over 1000 basepair sequence, but it does not contain any translational STOP codon. Xn is mononucleotide repeat with 5 to 50 identical nucleotides, and it is out-of-frame (3n+1 or 3n+2) with regard to the translational frame of the ORF following it. Xn can be polyguanie or polycytosine (e.g. G₂₂ or C₂₂). Open Reading Frame (ORF) is a protein coding sequence such as a fluorescent protein (XFP) or an immunoreporter (e.g. smFP or tandem smFPs. A Membrane Insertion Signal is a short peptide sequence that can target the reporter to plasma membrane of a cell (e.g. a farnesylation signal). Translational Stop is a codon that terminate ribosomal translation, and it can be TGA, TAG or TAA. (B). Construct #2 has an endogenous promoter preceding Open Reading Frame 1 (ORF1) with its own translation start site (i.e. ATG). ORF1 lacks its own translational stop codon. ORF 1 is followed by a sequence flanked with a translational stop codon and a spacer sequence (3 nucleotide or longer), and this sequence cassette is flanked by two recombinase binding sites (RBS1 and RBS2). The RBS1 and RBS2 has similar design as described above in FIG. 1A. After RBS2, there is a mononucleotide repeat of 5 to 50 nucleotides, e.g. polycytosine (C₂₂) or polyguanine (G₂₂). The mononucleotide repeat length is selected so that ORF2 and ORF1 are not in the same open reading frame without a frameshift of the mononucleotide repeat. The ORF2 can be a protein coding sequence such as a fluorescent protein (XFP) or an immunoreporter (e.g. smFP or tandem smFPs. ORF2 has its own in-frame translational stop codon. (C). A schematic of MORF3 sequence. It is located at the murine Rosa26 locus. MORF has a strong CAG promoter, followed by two LoxP sites flanking a transcriptional STOP sequence (consisted of multiple polyA signal). After the LoxP-STOP-LoxP cassette, there is a translational start codon (ATG), followed by a spacer of two Myc-tag coding sequence preceding the polycytosine repeat of 22 cytosines. The ORF in MORF3 is two spaghetti monster (smFP_immunoreporter fusion protein, each with 10 epitope-tags inserted into a superfold GFP scaffold. In some embodiment, each smFP has up to 10 V5 epitopes. The tandem smFP is fused at its C-terminal with a farnesylation signal as membrane insertion signal. WPRE is a Woodchuck Hepatitis Virus (WHP) Posttranscriptional Regulatory Element.

FIGS. 2A-2E illustrate MORF Sparse Labeling Strategy and In Vitro Optimization. (FIG. 2A) The stochastic frameshift of mononucleotide repeat can act as a translational switch and the four different types of repeats encode distinct polyamino acids. (FIG. 2B) A general design of Cre-dependent MORF reporter mouse line. (FIG. 2C) Testing the expression level of GFP in transfected HEK293FT cells with four mononucleotide repeats fused to GFP reporters. ANOVA with Tukey's test (**p<0.01 versus ATG-GFP, #p<0.01 versus ATG-C₂₁-GFP). (FIG. 2D) GFP reporter expression after moving the G₂₁ repeat away from the translation start codon with one or two Myc-tag spacer sequence. ANOVA with Tukey's test (**p<0.01 versus ATG-GFP, #p<0.01 versus ATG-Myc-Myc-G₂₁-GFP). (FIG. 2E) GFP reporter expression when a two Myc-tag spacer is placed between the translation start site and C21 repeat. ANOVA with Tukey's test (**p<0.01 versus ATG-GFP, #p<0.01 versus ATG-Myc-Myc-C21-GFP). For C, D, and E, n=3 or more replicates. Error bars are +/−SEM.

FIG. 3. The MORF1 and TIGRE-MORF transgenic reporter lines for Cre-dependent sparse cell labeling with membrane-bound fluorescent proteins. (A) Genetic construct for the MORF1 mouse line. The mNeonGreen-F is targeted to membrane using a farnesylation signal. (B-E) Direct mNeonGreen fluorescence reveals sparsely labeled cells in tissue sections from MORF1/Ella-Cre mice. (F-H) Brain sections from MORF1/Pcp2-Cre mice showing sparse labeling of cerebellar Purkinje cells (PCs) and their axons and axon terminals. (I-K) D2 medium spiny neurons (MSNs) in MORF1/Drd2-Cre mice demonstrating labeling from dendritic spine (J) to axon terminals in the Globus Pallidus externus (GPe; K). (L) Genetic construct for TIGRE-MORF (Ai166) mouse line. (M and N) TIGRE-MORF/TH-Cre brain sections showing labeled neuronal cell bodies in the substantia nigra pars compacta (SNc; M) and axon projections towards the striatum (N). (O and P) Representative images TIGRE-MORF/Camk2a-CreERT2 double transgenic mice showing cortical labeling of pyramidal neurons (O), with boxed region magnified in (P) including labeling of axonal segment (arrows). See also FIGS. 9 and 10.

FIG. 4. MORF3 transgenic reporter lines with the tandem smFPV5 immunoreporter sparsely and brightly label Cre-defined immunostained. (A) Cre-dependent MORF3 transgene design includes a tandem fusion of two membrane-localized smFPV5 (td-smFPV5) for maximal immunofluorescent detection. (B-E) Cortical PNs in layer V labeled in MORF3/Rbp4-Cre mice (counterstained with NeuN and DAPI), which reveal cell bodies, axons, dendrites (D) and dendritic spines (E). (F-H) MORF3/Pcp2-Cre mice show sparse labeling of cerebellar Purkinje Cells (PCs; co-labeled with calbindin) (F), with bright labeling their cell body and dendrites (G), and dendritic spines (H). (I-M) Examples of parvalbumin-expressing interneurons in MORF3/PV-Cre mice. Brain sections are double stained for V5 (MORF reporter). PV neurons in the cortex (I-K) and striatum (L-M) are shown, with boxed regions showing magnified images of dendritic processes and thinner, beaded axons (K and M). (N-R) Imaging of MORF3 and somatostatin (SST)-expressing interneurons in MORF3/SST-Cre mice can be resolved to show detailed dendritic and axonal morphologies (boxed regions) in the cortex (N-P) and striatum (Q-R). See also FIGS. 11 and 12.

FIG. 5. MORF3 mice enable brain-wide sparse labeling of microglia and astrocytes to reveal their detail morphology. (A-P) Tamoxifen-induced (TAM; 100 mg/kg for 5 days) sparse labeling of microglia with MORF3 throughout the MORF3/Cx3cr1-CreERT2 brain, including the cortex (A-C), striatum (E-G), hippocampus (I-K), and cerebellum (M-O), allows for more detailed imaging of processes and end feet when compared to Iba1 immunostaining (boxed regions); MORF3-labeled microglia can be readily reconstructed in full 3D (D, H, L, and P). (Q-T) MORF3/Aldh1I1-CreERT2 induced with 100 mg/kg tamoxifen for 3 days sparsely labels astrocytes with MORF3 throughout the brain, including the cortex (Q-R; counterstained for S100β), corpus callosum (S; counterstained for GFAP), as well as Bergmann glia in the cerebellum (T; co-labeled with GFAP). See also FIG. 13.

FIG. 6. MORF3 is compatible with transmission electron microscopy enabling mesoscale to nanoscale imaging. (A) Fluorescent labeling of neurons and processes in neocortex of a MORF3/Rbp4-Cre double transgenic mouse perfused with 0.5% GA and 4% PFA in 0.1 M PB. (B) A labeled neuron with immunostaining of the soma, primary and basal dendrites, and dendritic spines. Arrows (white) point to axons in cortex. (C, D) Membrane HRP immunolabeling of a cortical pyramidal neuron (C) and a dendrite and dendritic spines (D) from a 1-μm-thick plastic section near the section processed for immunofluorescence (A, B). (E) Transmission electron micrograph of an HRP-immunolabeled soma and primary dendrite. The plasma membrane is prominently immunostained with an increased density of staining in the cytoplasm. The nucleus (n) is unstained. Arrowheads point to the labeled soma and proximal dendrite. (F) Membrane labeling is prominent at and near the somatic plasma membrane (indicated on E) consistent with the plasma membrane-targeting of MORF3. (G) Labeled thin, myelinated axon, similar to the ones in B (arrows). (H) High magnification of a labeled presynaptic terminal onto two unlabeled postsynaptic terminals (*). (I) High magnification of a labeled presynaptic terminal onto an unlabeled small dendrite (*), surrounded by lighter, unlabeled processes. The density of cytoplasmic immunolabeling appears to be consistent in the axons, dendrites, and dendritic spines. Synapses have a higher density of immunolabeling compared to the labeling in the cytoplasm. (J) High magnification of a labeled presynaptic terminal onto an unlabeled postsynaptic terminal (*). Arrowheads in electron micrographs (E-J) point to immunolabeling. Scale bars=100 μm (A), 20 μm (B, C), 10 μm (D), 5 μm (E, F), and 500 nm (G, H, I, J).

FIG. 7. An imaging pipeline to capture the full dendritic morphologies of MORF-labeled neurons in thick brain sections and modulating the labeling frequency of MORF3 with tamoxifen-inducible Cre lines. (A) An imaging pipeline to immunolabel with anti-V5, clearing 500-600 μm thick brain sections with iDISCO+, image at 10× and 30× on the Andor DragonFly, and digitally reconstruct the MORF-labeled brain cells. (B) Sagittal brain sections from MORF3/CamK2a-CreERT2 mice processed with the iDISCO+ and imaged at 30× on the Andor DragonFly can capture the 3D dendritic morphology of multiple neuronal cell types brainwide. (C) The 3D dendritic morphology of individual MSNs can be imaged and digitally reconstructed. (D) Quantification of the number of primary dendritic branches extending from the cell body and (E) the length of the longest primary dendritic branch and the widest radius of the dendritic field in MORF3/CamK2a-CreERT2-labeled MSNs (n=40) from 500 μm thick sections. (F) Dose-dependent induction of Cre-recombinase expression with tamoxifen (0, 25, 50, and 100 mg/kg TAM for 1 day) in MORF3/Camk2a-CreERT2 mice increases the MSN labeling frequencies. (G-H) With a labeling frequency of 0.2% MSNs (tamoxifen induction with 25 mg/kg for 1 day) in the MORF3/Camk2a-CreERT2 striatum, the labeled MSNs can be readily reconstructed with our current pipeline (boxed region magnified in H). (I-L) The lowered labeling frequency of layer 5 cortical PNs in MORF3/Etv1-CreERT2 mice (100 mg/kg TAM for 3 days; I) enables the high-resolution imaging (J; boxed region magnified in L) and digital reconstruction (K) of individual neurons. (M-P) Similarly, regulating the labeling frequency of layer 2/3/4 cortical PNs in MORF3/Cux2-CreERT2 mice with tamoxifen (50 mg/kg for 1 day) enables detailed imaging (N; boxed region magnified in P) and digital reconstruction (O) of individual neurons. See also FIGS. 14-16 and Movies S2 and S3 (see online publication).

FIG. 8. Morphological Analysis of 151 MORF3-labeled Murine Retinal Horizontal Cells in Development. (A) Imaging and reconstructing the complete morphology of developing retinal horizontal cells in the postnatal day 5 retina of MORF3/Cx57-iCre mice. Dendritic field size is exemplified in the final panel of A with shading. (B) Correlation heatmap of morphological and anatomical features extracted from reconstructed 151 retinal horizontal cells. Blue and red signify a positive and negative correlations, respectively. Correlations above 0.2 in absolute value are shown explicitly. Clustering tree on the left is based on dissimilarity equal to one minus the correlation matrix. (C) T-distributed Stochastic Neighbor Embedding (t-SNE) plot of clustered retinal horizontal cells. Colors and shapes represent cell clusters. (D) Hierarchical cell clustering based on morphological features and soma locations extracted from retinal horizontal cells. The first row below the clustering tree represents clusters (each cluster is assigned a numeric label and color; label 0 and grey color are reserved for cells that are not part of a cluster); the rest of the rows represent, in heatmap form, cell shape statistics. For the heatmap, the shape statistics have been scaled to mean 0 and variance 1. The shape statistics' clustering tree on the left represents the same clustering as in panel B. (E) Clusters ordered by farthest soma distance from the center of the retina (least mature at periphery to most mature at center) with a representative example of neurons from each module. (F) Examples of retinal horizontal cells with more than one axonal projection or secondary process. See also FIGS. 18-20.

FIG. 9. Cre-mediated induction of the MORF1 transgene with AAV-Cre or Rgs9-Cre results in bright and sparse labeling within the basal ganglia. Related to FIG. 2.

FIG. 10. MORF2 transgenic reporter lines brightly and stochastically label immunostained neurons with the smFPV5. Related to FIG. 4.

FIG. 11. Digital reconstructions of the dendritic morphologies of MORF3 labeled cerebellar Purkinje cells, parvalbumin (PV)- and somatostatin (SST)-expressing interneurons. In B, brighter staining is Calb, and darker staining is V5. In E, brighter staining is V5, and darker staining is PV. In L, brighter staining is V5, and darker staining is SST. Related to FIG. 4.

FIG. 12. High-resolution images of MORF3/Drd2-Cre coronal sections. Related to FIG. 4.

FIG. 13. MORF3 labeling of microglia and astrocytes with tamoxifen-inducible CreERT2 lines. Related to FIG. 5.

FIG. 14. Comparison of Olympus silicone immersion objectives for imaging full morphology of MORF-labeled neurons in thick-sectioned, iDISCO+ cleared tissue. Related to FIG. 7.

FIG. 15. The labeling frequency of striatal MSNs in MORF3/Camk2a-CreERT2 can be tuned with tamoxifen to allow automated digital reconstruction and segmentation. Related to FIG. 4.

FIG. 16. The labeling frequency of cortical projections neurons can be tuned down with tamoxifen to allow digital reconstruction in MORF3 mice crossed with various layer-specific cortical CreERT2-lines. Related to FIG. 7.

FIG. 17. Sparse immunolabeling of striatal D1- and D2-MSNs at P0 in MORF3/Drd1-Cre and MORF3/Drd2-Cre mice, respectively. Related to FIG. 7.

FIG. 18. Retina whole mount of MORF3/Cx57-iCre at P5 demonstrating sparse, complete, and mostly non-overlapping labeling of retinal horizontal cells. Related to FIG. 8.

FIG. 19. Summary of single cell morphology of MORF3/Cx57-iCre retinal horizontal cells at P5. Related to FIG. 8.

FIG. 20. Concordance plots of morphological features extracted from reconstructed MORF3/Cx57-iCre retinal horizontal cells. Related to FIG. 8.

### DETAILED DESCRIPTION

The constructs and methods described herein are based on a discovery that greatly expands the capability for brainwide sparse labeling of genetically-defined neurons and glia for detailed analysis of their morphologies. Moreover, the tools and methods described herein provide the first simple, generalizable, and scalable solution for genetically-directed sparse cell labeling to systematically study brain cell morphology in the mammalian brain. The materials and methods described herein provide a simple, general and scalable solution in a mammalian species for genetically-directed sparse cell labeling that allows the visualization of the complete cellular morphology of cells. Representative examples of cells to be visualized using sparse labeling include, but are not limited to, neurons or non-neuronal cells in the central nervous system (including brain), peripheral nervous systems, and other peripheral tissues.

### Definitions

All scientific and technical terms used in this application have meanings commonly used in the art unless otherwise specified. As used in this application, the following words or phrases have the meanings specified.

As used herein, “complete morphology” in the context of a neuron, for example, includes axonal processes and dendrites, and in the cases of certain neurons, the presynaptic and postsynaptic terminals, in addition to the soma.

As used herein, a “significant difference” means a difference that can be detected in a manner that is considered reliable by one skilled in the art, such as a statistically significant difference, or a difference that is of sufficient magnitude that, under the circumstances, can be detected with a reasonable level of reliability. In one example, an increase or decrease of 10% relative to a reference amount is a significant difference. In other examples, an increase or decrease of 20%, 30%, 40%, or 50% relative to the reference is considered a significant difference. In yet another example, an increase of two-fold relative to a reference is considered significant.

“Nucleotide sequence” refers to a heteropolymer of deoxyribonucleotides, ribonucleotides, or peptide-nucleic acid sequences that may be assembled from smaller fragments, isolated from larger fragments, or chemically synthesized de novo or partially synthesized by combining shorter oligonucleotide linkers, or from a series of oligonucleotides, to provide a sequence which is capable of expressing the encoded protein.

As used herein, “hybridizes,” “hybridizing,” and “hybridization” means that the oligonucleotide forms a noncovalent interaction with the target DNA molecule under standard conditions. Standard hybridizing conditions are those conditions that allow an oligonucleotide probe or primer to hybridize to a target DNA molecule. Such conditions are readily determined for an oligonucleotide probe or primer and the target DNA molecule using techniques well known to those skilled in the art. The nucleotide sequence of a target polynucleotide is generally a sequence complementary to the oligonucleotide primer or probe. The hybridizing oligonucleotide may contain nonhybridizing nucleotides that do not interfere with forming the noncovalent interaction. The nonhybridizing nucleotides of an oligonucleotide primer or probe may be located at an end of the hybridizing oligonucleotide or within the hybridizing oligonucleotide. Thus, an oligonucleotide probe or primer does not have to be complementary to all the nucleotides of the target sequence as long as there is hybridization under standard hybridization conditions.

The term “complement” and “complementary” as used herein, refers to the ability of two DNA molecules to base pair with each other, where an adenine on one DNA molecule will base pair to a guanine on a second DNA molecule and a cytosine on one DNA molecule will base pair to a thymine on a second DNA molecule. Two DNA molecules are complementary to each other when a nucleotide sequence in one DNA molecule can base pair with a nucleotide sequence in a second DNA molecule. For instance, the two DNA molecules 5′-ATGC and 5′-GCAT are complementary, and the complement of the DNA molecule 5′-ATGC is 5′-GCAT. The term complement and complementary also encompasses two DNA molecules where one DNA molecule contains at least one nucleotide that will not base pair to at least one nucleotide present on a second DNA molecule. For instance, the third nucleotide of each of the two DNA molecules 5′-ATTGC and 5′-GCTAT will not base pair, but these two DNA molecules are complementary as defined herein. Typically, two DNA molecules are complementary if they hybridize under the standard conditions referred to above. Typically, two DNA molecules are complementary if they have at least about 80% sequence identity, preferably at least about 90% sequence identity.

As used herein, the term “subject” includes any human or non-human animal. The term “non-human animal” includes all vertebrates, e.g., mammals and non-mammals, such as non-human primates, horses, sheep, dogs, cows, pigs, chickens, and other veterinary subjects. In a typical embodiment, the subject is a mouse.

As used herein, “a” or “an” means at least one, unless clearly indicated otherwise.

### Nucleic Acid Constructs

In one embodiment, described herein is a nucleic acid construct comprising, in operable linkage, a translation start site, an optional spacer, a polycytosine or polyguanine mononucleotide repeat, and an open reading frame (ORF), wherein the polycytosine or polyguanine repeat and the ORF are out of frame with respect to the translation start site.

In some embodiments, the mononucleotide repeat is a polycytosine repeat. In some embodiments, the polycytosine mononucleotide repeat consists of a minimum of 5 cytosines. In some embodiments, the polycytosine mononucleotide repeat consists of 5 to 25 cytosines. In some embodiments, the polycytosine mononucleotide repeat consists of 5 to 50 cytosines. In some embodiments, the polycytosine mononucleotide repeat consists of 22 cytosines (C₂₂). In some embodiments, the spacer comprises at least 3 base pairs, but it does not contain a translational STOP codon (i.e. TAG, TGA or TAA). In some embodiments, the spacer comprises between 3 and 1000 base pairs, but it does not contain a translational STOP codon (i.e. TAG, TGA or TAA). In some embodiments, the spacer comprises at least 3 and can be over 1000 base pairs, but it does not contain a translational STOP codon (i.e. TAG, TGA or TAA). In some embodiments, the spacer comprises between 30 and 100 base pairs. In some embodiments, the optional spacer sequence encodes one or two Myc tags.

In some embodiments, the ORF encodes a fluorescent protein. In some embodiments, the fluorescent protein is GFP, RFP, tdTomato, and/or mNeonGreen. In some embodiments, the ORF encodes an immunoreporter (e.g. an immunoreactive epitope tag-containing protein). In some embodiments, the ORF encodes a spaghetti monster immunoreporter with up to 10 epitope-tags inserted into a superfold GFP scaffold. In some embodiments, the immunoreporter comprises simian virus 5-derived epitope (V5), myelocytomatosis viral oncogene (Myc), hemagglutinin (HA), a FLAG tag, and/or OLLAS (Escherichia coli OmpF linker and mouse langerin). In some embodiments, the immunoreporter is a spaghetti monster immunoreporter with V5 epitope tags. Spaghetti monster fluorescent proteins are discussed in Viswanathan et al., 2015, Nature Methods 12(6): 568.

In some embodiments, the ORF encodes a functional protein that can activate or suppress gene expression, such as site-specific recombinase (e.g. Cre, Flp or FlpO, Dre, or Vika) or site-specific integrase (e.g. PhiC31, Bxb1). In some embodiments, the ORF encodes a genetically-encoded calcium indicator or “GECI” (e.g. GCaMP3, GCaMP5, GCaMP6, jGCaMP7, or R-GRECO1). In some embodiments, the ORF encodes one or more DNA or RNA programmable nucleases that enable genomic DNA or RNA editing (e.g. SpCas9, SaCas9 Cpf1, Cas13, ZFNs, TALENs).

In some embodiments, the ORF encodes a tandem fusion of two spaghetti monster immunoreporters with each immunoreporter consisting of up to 10 epitope tags inserted into the superfold GFP scaffold. In some embodiments, the ORF encodes a tandem fusion of two spaghetti monster immunoreporters with a total of up to 20 V5 epitope tags. In some embodiments, the ORF encodes a tandem fusion of two immunoreporters with a total of up to 20 epitope tags, which may include, but is not limited to, any combination of Myc tags, HA tags, FLAG tag, V5 tags, and OLLAS tag.

In some embodiments, the ORF encodes a membrane insertion signal. In some embodiments, the ORF is fused at the C-terminal with a farnesylation signal. In some embodiments, the farnesylation signal is a Ras CAAX domain.

In some embodiments, the ORF encodes a polypeptide or protein that has enzymatic activity. Examples of such polypeptides and proteins having enzymatic activity include those described in the following references: APEX labeling and BioID: Rhee, H. W. et al. Proteomic mapping of mitochondria in living cells via spatially restricted enzymatic tagging. Science 339, 1328-1331 (2013); Lam, S. S. et al. Directed evolution of APEX2 for electron microscopy and proximity labeling. Nat. Methods 12, 51-54 (2015); Li P., Li J., Wang L. & Di L. J. Proximity labeling of interacting proteins: application of BioID as a discovery tool. Proteomics 17, 10.1002/pmic.201700002 (2017); Roux, K. J., Kim, D. I., Raida, M. & Burke, B. A promiscuous biotin ligase fusion protein identifies proximal and interacting proteins in mammalian cells. J. Cell Biol. 196, 801-810 (2012).

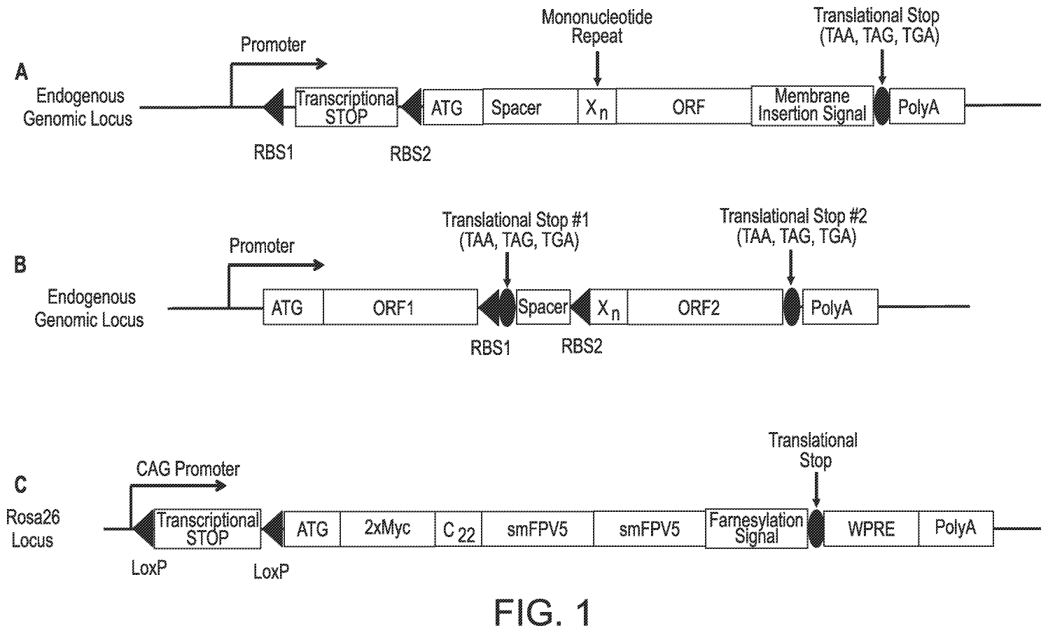

In some embodiments, the construct further comprises a polyadenylation signal downstream from the ORF. In some embodiments, the construct further comprises a protein coding sequence between the translation start site (ATG) and the polycytosine mononucleotide repeat. In some embodiments, the construct further comprises a protein coding sequence inserted between the polycytosine mononucleotide repeat and the ORF. In some embodiments, the protein coding sequence is a cDNA or a genomic DNA. In some embodiments, the construct is that illustrated in FIG. 1. In some embodiments, the construct is that of FIG. 1A; in some embodiments, the construct is that of FIG. 1B; and in some embodiments, the construct is that of FIG. 1C.

In some embodiments, the construct further comprises a promoter, a transcriptional stop sequence, and two site-specific recombinase binding sites flanking the transcriptional stop sequence, wherein the promoter is upstream of the recombinase binding sites, and wherein each of the preceding elements is upstream of the translation start site. In some embodiments, the promoter is a cytomegalovirus early enhancer element and/or a chicken beta actin (CAG) promoter. In some embodiments, the transcriptional stop sequence contains at least one polyadenylation signal. In some embodiments, the recombinase binding sites are LoxP sites, and the LoxP sites are oriented such that Cre recombinase excises the transcriptional stop sequence. In some embodiments, the recombinase binding sites are Frt sites, and the Frt sites are oriented such that Flp recombinase excises the transcriptional stop sequence. In some embodiments, the recombinase binding sites are Vox sites, and the Vox sites are oriented such that Vika recombinase excises the transcriptional stop sequence. In some embodiments, the recombinase binding sites are Rox sites, and the Rox sites are oriented such that Dre recombinase excises the transcriptional stop sequence. In some embodiments, the recombinase binding sites are Vox sites, and the Vox sites are oriented such that Vika recombinase excises the transcriptional stop sequence. In some embodiments, the recombinase binding sites comprise one attB site and one attP site, which are specific for PhiC31 integrase. The attB and attP sites are oriented such that PhiC31 integrase excises the transcriptional stop sequence. In some embodiments, the recombinase binding sites comprise one attB site and one attP site, which are specific for BxB1 integrase. The attB and attP sites are oriented such that Bxb1 integrase excises the transcriptional stop sequence.

Also described is a nucleic acid construct comprising, in operable linkage, a translation start site, a spacer, a polyguanine mononucleotide repeat, an obligatory spacer of at least 3 base pairs, and an open reading frame (ORF), wherein the polyguanine mononucleotide repeat and the ORF are out of frame with respect to the translation start site. In some embodiments, the polyguanine mononucleotide repeat consists of at least 5 guanines. In some embodiments, the polyguanine mononucleotide repeat consists of between 5 and 25 guanines. In some embodiments, the polyguanine mononucleotide repeat consists of between 5 and 50 guanines. In some embodiments, the polyguanine mononucleotide repeat consists of 22 guanines (G₂₂). In some embodiments, the spacer comprises between 3 and 1000 base pairs. In some embodiments, the spacer comprises at least 30 base pairs. In some embodiments, the spacer sequence encodes one or two Myc tags.

In some embodiments, the ORF encodes a fluorescent protein. In some embodiments, the fluorescent protein is GFP, RFP, tdTomato, and/or mNeonGreen. In some embodiments, the ORF encodes an immunoreporter, such as, for example, an immunoreactive epitope tag-containing protein. In some embodiments, the ORF encodes a spaghetti monster immunoreporter with up to 10 epitope-tags inserted into a superfold GFP scaffold. In some embodiments, the immunoreporter comprises simian virus 5-derived epitope (V5), myelocytomatosis viral oncogene (Myc), hemagglutinin (HA), FLAG tag, and or OLLAS (Escherichia coli OmpF linker and mouse langerin). In some embodiments, the immunoreporter is a spaghetti monster immunoreporter with V5 epitope tags.

In some embodiments, the ORF encodes a tandem fusion of two spaghetti monster immunoreporters with each immunoreporter consisting of up to 10 epitope tags inserted into the superfold GFP scaffold. In some embodiments, the ORF encodes a tandem fusion of two spaghetti monster immunoreporters with a total of up to 20 V5 epitope tags. In other embodiments, the ORF encodes a tandem fusion of two immunoreporters with a total of up to 20 epitope tags, which maybe include but not limited to any combination of Myc tags, HA tags, FLAG tag, V5 tags, and or OLLAS (Escherichia coli OmpF linker and mouse langerin).

In some embodiments, the ORF encodes a membrane insertion signal. In some embodiments, the ORF is fused at the C-terminal with a farnesylation signal. In some embodiments, the farnesylation signal is a Ras CAAX domain. In some embodiments, the ORF encodes a polypeptide or protein that has enzymatic activity.

In some embodiments, the construct further comprises at least one polyadenylation signal downstream from the ORF.

In some embodiments, the DNA construct has two open reading frames in the following configurations. First, a promoter preceding the Open Reading Frame 1 (ORF1), which is followed by a mononucleotide repeat, which in turn is followed by an Open Reading Frame 2 (ORF2). In some embodiments, the mononucleotide repeats comprise polyguanine with 5 to 50 guanine nucleotide residues or polycytosine with 5 to 50 cytosine nucleotide residues. The number of nucleotides is selected so that the ORF2 is not in the same open reading frame as ORF1 without a frameshift of the mononucleotide repeat.

In some embodiments, the ORF1 in the place of spacer is a cDNA or a genomic DNA. In some embodiments, this genomic DNA is an endogenous genomic DNA that encodes a protein. In some embodiments, a promoter is placed in front of the ORF1.

In some embodiments, the promoter is a cytomegalovirus early enhancer element and chicken beta actin (CAG) promoter.

In some embodiments, a translational stop codon flanked by two recombinase binding sites is placed between the ORF1 and the mononucleotide repeat, which is followed by a second open reading frame (i.e. ORF2). In some embodiments, the translational stop sequence is followed by a spacer sequence of 3 nucleotides or more, and the translation stop plus spacer cassette is flanked by two recombinase binding sites. The number of nucleotides is selected so that the ORF2 is not in the same open reading frame as in ORF1 without a frameshift of the mononucleotide repeat. Moreover, the space sequence as well as the recombinase binding sites are designed so that there will be no translational stop codon between ORF1 and ORF2 after at least one of the frameshifts of the mononucleotide repeat.

In some embodiments, the recombinase binding sites are LoxP sites, and the LoxP sites are oriented such that Cre recombinase excises the translational stop sequence. In some embodiments, the recombinase binding sites are Frt sites, and the Frt sites are oriented such that Flp recombinase excises the translational stop sequence. In some embodiments, the recombinase binding sites are Vox sites, and the Vox sites are oriented such that Vika recombinase excises the translational stop sequence. In some embodiments, the recombinase binding sites are Rox sites, and the Rox sites are oriented such that Dre recombinase excises the translational stop sequence. In some embodiments, the recombinase binding sites are Vox sites, and the Vox sites are oriented such that Vika recombinase excises the translational stop sequence. In some embodiments, the recombinase binding sites are consisted of one attB site and one attP site, which are specific for PhiC31 integrase. The attB and attP sites are oriented such that PhiC31 integrase excises the translational stop sequence. In some embodiments, the recombinase binding sites are consisted of one attB site and one attP site, which are specific for BxB1 integrase. The attB and attP sites are oriented such that Bxb1 integrase excises the translational stop sequence. In all cases, the recombinase mediated excision of the translational stop sites do not introduce additional translational stop sequence between the ORF and the mononucleotide repeats.

In some embodiments, the ORF2 encodes a fluorescent protein. In some embodiments, the fluorescent protein is GFP, RFP, tdTomato, and/or mNeonGreen. In some embodiments, the ORF2 encodes an immunoreporter, such as, for example, an immunoreactive epitope tag-containing protein. In some embodiments, the ORF2 encodes a spaghetti monster immunoreporter with up to 10 epitope-tags inserted into a superfold GFP scaffold. In some embodiments, the immunoreporter comprises simian virus 5-derived epitope (V5), myelocytomatosis viral oncogene (Myc), hemagglutinin (HA), FLAG tag and or OLLAS (Escherichia coli OmpF linker and mouse langerin). In some embodiments, the immunoreporter is a spaghetti monster immunoreporter with V5 epitope tags.

In some embodiments, the ORF2 encodes a tandem fusion of two spaghetti monster immunoreporters with each immunoreporter consisting of up to 10 epitope tags inserted into the superfold GFP scaffold. In some embodiments, the ORF2 encodes a tandem fusion of two spaghetti monster immunoreporters with a total of up to 20 V5 epitope tags. In other embodiments, the ORF2 encodes a tandem fusion of two immunoreporters with a total of up to 20 epitope tags, which maybe include but not limited to any combination of Myc tags, HA tags, FLAG tag, V5 tags, and or OLLAS (Escherichia coli OmpF linker and mouse langerin).

### Cells

Also provided is a cell comprising a construct as described herein. In some embodiments, the cell is a vertebrate cell. In some embodiments, the cell is a mammalian cell. Examples of mammalian cells include, but are not limited to, a murine cell. Additionally provided is an animal comprising a cell as described herein. In some embodiments, the animal is murine (rodents, such as mice, rats), avian (chicken, turkey, fowl), bovine (beef, cow, cattle), ovine (lamb, sheep, goats), porcine (pig, swine), piscine (fish), non-human primates (e.g. marmosets), or other vertebrates (e.g. frogs, fishes). In a preferred embodiment, the animal is a rodent, such as a mouse or a rat.

### Methods

Described herein is a method of producing sparse and stochastic labeling of Cre-expressing cells in a host mammal, the method comprising generating a mouse that expresses the construct as described herein. In some embodiments, the labeling reveals the complete morphology of the cells. For neurons and other specialized cell types, the complete morphology can include processes that extend from the cell, such as axons and dendrites. The method employs optimized constructs that overcome limitations of prior attempts at developing sparse and stochastic labeling of cells, allowing for visualization of full neuronal morphology from dendrites and axons to spines. The exemplary mouse cell types described herein utilize membrane-bound, direct fluorescent reporters and immune-reporters to illuminate morphologies of genetically-defined neurons. This provides a scalable platform to capture the three-dimensional morphologies of individual neurons for extensive analyses.

By introducing a stochastic translational switch (polycytosine or polyguanine repeats), one can achieve sparse expression of a desired gene. This technique can be applied to transgenes (i.e. exogenously inserted genes into the genome) as well as to endogenous genes. In the case of endogenous genes, the translation switch (polycytosine or polyguanine repeat) can be used to sparsely and stochastically switch on the expression of a fluorescent, immunoreporter or enzymatically active protein as a fusion protein to an endogenously expressed protein in a cell.

### Kits

The invention provides kits comprising a set of reagents as described herein, such as nucleotide sequences that comprise constructs of the invention and other elements for use in preparing such constructs or for use in practicing methods described herein, and optionally, one or more suitable containers containing reagents of the invention. Reagents can optionally include a detectable label. Labels can be fluorescent, luminescent, enzymatic, chromogenic, or radioactive.

Kits can include probes for detection of expression products in addition to antibodies for protein and epitope detection. The kit can optionally include a buffer. Reagents and standards can be provided in combinations reflecting the combinations of elements described herein as useful.

### EXAMPLES

The following examples are presented to illustrate the present invention and to assist one of ordinary skill in making and using the same. The examples are not intended in any way to otherwise limit the scope of the invention.

### Example 1: Genetically-Directed Sparse Neuronal Labeling in Transgenic Mice Through Monocluotide Repeat Frameshift

This Example demonstrates that Mosaicism with Repeat Frameshift (MORF) allows a single Bacterial Artificial Chromosome (BAC) transgene to direct sparse labeling of genetically-defined neuronal populations in mice. The BAC transgene drives cell-type-specific transcription of an out-of-frame mononucleotide repeat that is placed between a translational start codon and a membrane-bound fluorescent protein lacking its start codon. The stochastic frameshift of the unstable repeat DNA in a subset of BAC-expressing neurons results in the in-frame translation of the reporter protein hence the sparse neuronal labeling. As a proof-of-concept, we generated D1-dopamine receptor (D1) BAC MORF mice that label about 1% striatal D1-expressing medium spiny neurons and allow visualization of their dendrites. These mice enable the study of D1-MSN dendrite development in wildtype mice, and its degeneration in a mouse model of Huntington's disease.

Details of this Example can be found in Lu and Yang, 2017, Sci Rep. 7, 43915. This mouse model, however, has notable limitations. The labeling brightness in D1-BAC-MORF mice is weak and requires signal amplification for detection. Moreover, the synapses and axons of the labeled neurons are partial, and it is unclear if the MORF reporters can be improved to achieve more complete yet non-toxic and sparse labeling of neurons in the brain. Finally, it remains unknown whether the MORF approach can be generally applied to sparsely label different neuronal cell types in the brain or other non-neuronal cell types in the mouse.

### Example 2: Optimizing Mononucleotide Repeat Frameshift Mice to Illuminate the Complete Morphology of Genetically-Defined Neurons in the Brain

This Example describes the stepwise optimization of reporter mice conferring Cre-dependent sparse cell labeling based on mononucleotide repeat frameshift (MORF) as a translational switch. All three MORF mouse lines label 1-5% of Cre-defined cell populations, and optimization led to brighter, non-toxic, and generalizable labeling of full neuronal morphologies including dendrites, axons, and synapses. Notably, the optimized MORF3 mice with a novel multivalent immunoreporter confers orders-of-magnitude brighter signal than the baseline MORF1 mice. MORF3 mice are compatible with mesoscale imaging of intact dendritic morphology in tissue-cleared thick brain sections as well as nanoscale imaging of neuronal ultrastructure with immune-EM. Finally, tissue-wide imaging, full morphological reconstruction, and unbiased analyses of 151 MORF3-labeled developing retinal horizontal cells identified cell clusters with novel patterns of axonal growth and maturation. Together, this study demonstrates a conceptually novel mouse genetic solution to sparsely label and illuminate the complete morphology of genetically-defined neurons in the intact mammalian brain.

The study upon which this Example is based has been published as Veldman, M. B., et al., “Brainwide Genetic Sparse Cell Labeling to Illuminate the Morphology of Neurons and Glia with Cre-Dependent MORF Mice” Neuron 108(1): 14 Oct. 2020, Pages 111-127. The online publication of this article contains the original figures and movies referenced herein.

Materials Availability

Three newly generated MORF mouse lines are deposited at the Jackson Laboratory for distribution to the scientific community. The JAX catalog number for these mice are: JAX #035400 (MORF1), JAX #035403 (MORF3), and JAX #035404 (TIGRE-MORF/Ai166).

Experimental Model and Subject Details

Animal Care and Use

Animals were housed in a specific-pathogen-free barrier facility, with up to four mice per cage with food and water available ad libitum. They were housed in a temperature-controlled environment with 12-hour light/dark cycle. Mouse care in the current study was in accordance with the United States Public Health Service Guide for the care and Use of Laboratory Animals. The procedures were approved by Chancellor's Animal Research Committee (ARC) at UCLA. Veterinarian care was provided by the UCLA Division of Laboratory Animal Medicine. Sex matched animals were used for all experiments. No obvious difference between sexes were noted.

Method Details

In Vitro Optimization of MORF Expression

Plasmids harboring the described mononucleotide repeats and fluorescent reporters were cloned using standard molecular cloning techniques. The pCS2+ plasmid backbone was used for transient transfection reporter experiments. Experimental GFP reporter plasmids were co-transfected with pCS2-mCherry reporter for normalization of transfection efficiency into HEK293FT cells using FuGENE HD reagent (Promega, E2311). Cells were imaged 48 hours post transfection with standardized, non-saturating exposure times to measure fluorescent brightness. Unmodified GFP reporter was set at 100% brightness and experimental plasmid brightness normalized accordingly. Transfections were repeated at least three times and measurements for each field and channel were taken from three random fields per transfection using the ImageJ mean grey value function.

Transgenic Mouse Generation

MORF1 and MORF3 transgenic lines were generated through traditional Rosa26 gene knockin methods performed at the University of California-Irvine Transgenic Mouse Facility. The MORF1 and the MORF3 reporters were cloned using standard molecular cloning techniques into the Ai9 plasmid (Madisen et al., 2010), obtained from Addgene (plasmid #22799), at the FseI restriction sites to replace the original tdTomato reporter gene. The MORF1 reporter consists of a Kozak consensus start site followed by two Myc-tags, C₂₂-repeat, mNeonGreen fluorescent reporter (Shaner et al., 2013), and membrane localization signal CAAX domain from Ras. The MORF3 reporter consists of a Kozak consensus start site followed by two Myc-tags, C₂₂-repeat, the first smFP V5 reporter, tdTomato linker domain sequence, the second smFP V5 reporter, and membrane localization signal CAAX domain from Ras. smFP V5 was cloned from pCAG_smFP V5 plasmid obtained from Addgene (plasmid #59758). MORF2 transgenic mice were generated using the ϕC31 integrase method of targeted transgene insertion into the Rosa26 locus (Tasic et al., 2011) performed by Applied StemCell. The MORF2 reporter harboring a Kozak consensus start site followed by two Myc-tags, C₂₂-repeat, mNeonGreen fluorescent reporter, tdTomato linker domain, smFP V5 reporter, and membrane localization signal CAAX domain from Ras was cloned into the pTARGATT6 vector for gene knockin. The TIGRE-MORF mouse line was generated using recombinase-mediated cassette exchange as described previously for TIGRE2.0 (Daigle et al., 2018). The TIGRE-MORF reporter harbors a Kozak consensus start site followed by two Myc-tags, G₂₂-repeat, EGFP reporter, and membrane localization signal CAAX domain from Ras cloned into Flp targeted vector p841 TIGRE FlpIn. Germline transmission of each transgene was confirmed by PCR and adhered to the predicted Mendelian inheritance ratios. Mice were maintained on the C57BL/6J background.

Stereotaxic Viral Injections

Mice were deeply anesthetized with isofluorane (1-2%) and mounted onto a stereotaxic frame with non-puncturing ear bars (Kopf Instruments). The scalp was opened and a hole for unilateral injection was drilled with a 0.5 mm burr drill bit at the site of injection above the dorsal striatum (coordinates from bregma; AP=+1.0 mm, ML=+1.6 mm). rAAV2/2.CMV.Cre (1.1e12 GC/ml injection titer; University of Iowa Gene Transfer Vector Core) was unilaterally injected into the dorsal striatum (coordinates from surface of the brain; DV=−2.5 mm) through a 33-gauge injector cannula (PlasticsOne) using a syringe pump (KDS) at a rate of 0.2 μl/min (0.5 μl total). After viral injection, the scalp was carefully closed and sutured. Following the surgical procedure, mice were individually housed and monitored for body weight and health until recovery from the surgery (1 week). To allow enough time for viral expression post-injection, we waited 2-3 weeks before perfusing the mice for tissue collection.

Tamoxifen Induction of CreERT2 Mice

Tamoxifen stock was prepared by dissolving tamoxifen (Cayman Chemical; Cat #13258) in corn oil (Sigma-Aldrich; Cat #C8267) at a concentration of 20 mg/ml by shaking overnight at 37° C. Tamoxifen was adjusted with corn oil to suitable working concentrations to administer by intraperitoneal (i.p.) injection at the doses stated (25-100 mg/kg body weight in approximately 200 μl of injection volume). CreERT2 mice received i.p. injections of tamoxifen once per day for 1 to 5 days (as indicated in text) and tissue was collected 1 week after the final injection.

Immunostaining and Imaging of Brain Sections

Mice were transcardially perfused with 50 mL of 0.1 M phosphate buffered solution (PBS) followed by 50 mL of ice cold 4% paraformaldehyde (PFA). Tissues were then dissected and post-fixed in PFA overnight at 4° C. For cryosectioning, tissues were incubated in 30% sucrose PBS for 48 hours and then snap frozen in powdered dry ice. Tissues were then sectioned at 40 μm on a cryostat. Immunostaining was performed using a standard protocol for floating sections. Sections were rinsed 3 times in PBS then blocked in 3% bovine serum albumin and 3% normal goat serum with 0.1% Triton X-100 for 1 hour. Primary antibodies at the noted concentrations were added to the blocking solution and sections incubated overnight at 4° C. with gentle shaking. The following day, sections were washed 3 times for fifteen minutes each and then incubated in secondary antibody (1:500 dilution for all secondaries) for at least 2 hours at room temperature. Sections were then stained with DAPI to label nuclei for 10 minutes, washed 3 times for ten minutes each, and finally mounted on slides with Prolong Diamond antifade reagent (P36965, ThermoFisher). Following curing overnight, slides were imaged using a Zeiss LSM800 confocal microscope. The following primary antibody dilutions were used: Mouse monoclonal anti-V5 tag (1:1000); Rabbit polyclonal anti-V5 tag (1:1000); Rabbit polyclonal anti-calbindin (1:1000); Mouse monoclonal anti-parvalbumin (1:3000); Rabbit polyclonal anti-DARPP-32 (1:500); Rabbit polyclonal anti-tyrosine hydroxylase (1:1000); and Mouse monoclonal anti-NeuN (1:1000).

Immunostaining and Imaging of Retina Wholemounts and Sections

Mice were euthanized with CO₂ inhalation and retinas were dissected for wholemounts as described in Dunn and Wong, 2012. Retinas were fixed with 4% PFA in PBS for 15 minutes at room temperature then rinsed with PBS before incubating with blocking reagent (5% Normal Goat Serum and 5% Triton-X in PBS) overnight at 4° C. After blocking, primary antibodies were added in blocking reagent and incubated for 5 days at 4° C. Then, retinas were washed 3×'s for 1 hour with PBS. Secondary antibodies were then added in blocking reagent and retinas were incubated overnight at 4° C. Retinas were then washed 3×'s for 1 hour with PBS and mounted onto filter paper and prepared for imaging. Primary antibodies were used at the following dilutions: Chicken polyclonal anti-V5 (1:500) and Rabbit polyclonal anti-Cone Arrestin (1:500). All secondary antibodies were used at 1:500 dilution. The Cx57-iCre/MORF3 mice were sacrificed at P5 and tissue prepared as detailed above. The prepared tissue was imaged on an Andor DragonFly (Belfast, UK) spinning disk confocal microscope with a 30× silicone oil objective at 1 μm z-steps running Andor Fusion 2.1 and visualized with Imaris 9.3.

For retina sections, eyes of euthanized mice were enucleated and fixed with 4% PFA in PBS for 1 hour on ice. The cornea and lens were removed following fixation and the remaining eye cups were placed in 30% sucrose overnight at 4° C. Eye cups were then frozen with OCT and retinas were sectioned at 20 μm with a cryostat. Alternating sections were collected in 10 different slides with each slide containing tissue representative of the entire retina. For immunostaining, slides were blocked with 10% Normal Goat Serum and 5% Triton-X in PBS for 1 hour at room temperature. Then, sections were incubated with primary antibody overnight at 4° C. They were then rinsed 3×'s with PBS for 15 minutes. Retina sections were then incubated with secondary antibody overnight at 4° C. These were subsequently washed 3×'s with PBS. They were then treated with DAPI (1:1000) for 30 minutes at room temperature and then rinsed 1× with PBS. Slides were then prepared for imaging. Primary antibodies were used at the following dilutions: Chicken polyclonal anti-V5 (1:500) and Rabbit polyclonal anti-calbindin (1:2000). All secondaries were used at 1:500 dilution.

Electron Microscopy

Mice were deeply anesthetized with pentobarbital and perfused transcardially with ice-cold 0.2% or 0.5% glutaraldehyde (GA) and 4% paraformaldehyde (PFA) in 0.1 M phosphate buffer (PB), pH 7.4. The brain was removed and post-fixed in 0.2% or 0.5% GA and 4% PFA in 0.1 M PB overnight at 4° C. The brain was washed in 0.1 M phosphate buffered saline (PBS, 0.1M, pH 7.4) and embedded in 10% gelatin (Sigma-Aldrich) dissolved in PBS. Vibratome sections were cut at 90 μm from the rostral forebrain through the hippocampus in the transverse plane. Sections were washed and then stored in 0.1 M PBS at 4° C.

Selected vibratome sections were 1) washed in 0.1 M PBS, 2) incubated in 1% sodium borohydride (NaBH4) for 30 minutes in 0.1 M PBS, or 3) immersed in 30% sucrose 0.1 M PBS and freeze thawed 3 times. The sections were washed in 0.1 M PBS for 3×10 minutes, transferred to plastic cell culture wells (NUNC) and immersed in a blocking solution (1% BSA fraction V, Sigma-Aldrich, 10% normal goat serum; NGS, S-1000, Vector Laboratories) for 1 hour, followed by incubation with the primary antibody (anti-V5 raised in rabbit, V8137, Sigma-Aldrich, for peroxidase) (1:500 in the blocking solution) for 5 days at 4° C. Thereafter, the sections were washed (3×10 minutes) and incubated in the secondary goat anti-rabbit IgG antibody labeled with horseradish peroxidase (HRP) (1:1000, in PBS, plus 3 drops of NGS, Vector Labs). Sections incubated in HRP were washed with PBS (3×10 minutes) and incubated in 3,3′-diaminobenzidine (DAB) solution (ImmPACT™ DAB, SK-4103, Vector Labs, peroxidase substrate kit), reacted for 5 minutes, washed 3 times 10 minutes with PBS, and processed for transmission electron microscopy. Sections processed for immunofluorescence were incubated in chicken polyclonal anti-V5 tag (1:500, Abcam) antibody in 1% BSA, 3% NGS, 0.1% NaN₃ in PBS for 3 days at room temperature with gentle shaking, washed 4 times for 15 minutes each in PBS, then incubated in Alexa 488 goat anti-chicken IgY (1:1000, Abcam) in 1% BSA, 3% NGS in PBS for 3 hours at room temperature with gentle shaking, washed in PBS 4 times for 15 minutes each, mounted in Prolong Gold antifade reagent (P36930, ThermoFisher), and evaluated using a Zeiss LSM880 confocal microscope.

The sections were washed in sodium cacodylate buffer (0.15 M, pH 7.4) 3 times for 20 minutes each, then immersed in 0.1% of aqueous osmium tetroxide (OsO4) for 30 minutes. Tissues were then rinsed with double distilled water (3×10 minutes) and placed in 1% uranyl acetate (aqueous) and stored overnight at 4° C. Tissue sections were washed with double distilled water (3×10 minutes) and dehydrated in ascending ethanol (30%, 50%, 70%, 80%, 95%, 2×, 100× for 10 minutes each), and 5 minutes propylene oxide. Sections were then infiltrated with resin Epon® 812 (Electron Microscopy Sciences (EMS), Hatfield, Pa., USA) as follows: a mixture of 100% resin and two parts of propylene oxide for two hours, and then 100% resin for 2 hours. Sections were flat mounted and then incubated in the oven (65° C.) with fresh resin for 48 hours. The areas of interest (cortex and hippocampus) were cut from the section (1×1 mm) and mounted on polymerized Epon blocks using superglue. Thin sections (0.5-micron thick, approximately 5-10 sections), were obtained to identify immunoreactive somata, dendrites, axons, and terminals. Ultrathin sections (90 nm thick) were obtained using a diamond knife (Diatome) with an AO/Reichter Ultracut-E microtome. Sections were collected on single slot carbon-formvar coated copper grids (0.5×2 mm).

Transmission electron microscopy observations and digital image captures were made using a FEI Tecnai transmission electron microscope T12 TEM −120 KV (Hillsboro, Oreg. USA). All sections were systematically analyzed at low (2000× magnification), and higher magnifications (4,000-10,000×). Sections were studied for the presence of immunoreactive somata, dendrites and axons, and terminals. Images were collected using a Gatan 2 k×2 k CCD camera at a 0.2 nm line resolution. Images were post-processed using Adobe Photoshop for adjustment of brightness and contrast.

iDISCO+ Tissue Immunostaining, Clearing, and Imaging of Thick Brain Sections

Mice were transcardially perfused with 50 mL of 0.1 M phosphate buffered solution (PBS) followed by 50 mL of ice cold 4% paraformaldehyde (PFA). Tissues were then dissected and post-fixed in PFA overnight at 4° C. Tissues were stored at 4° C. in 0.01M PBS with 0.02% sodium azide. Tissues were vibratome sectioned (Compresstome; Precisionary Instruments) at 300-600 μm. Immunostaining and clearing of the thick sections were adapted for MORF tissues from the iDISCO+ protocol (Renier et al., 2016).

Prior to immunolabeling, sections were dehydrated with a series of methanol (MeOH) washes (20%, 40%, 60%, 80%, 100%; room temperature for 1 hour at each step) and membrane lipids were removed with an overnight incubation in 66% dichloromethane (DCM) and 33% MeOH at room temperature. Sections were bleached with hydrogen peroxide (5% in MeOH) overnight at 4° C. to reduce tissue autofluorescence. Sections were then rehydrated with decreasing concentrations of MeOH (80%, 60%, 40%, 20%, then 0.01M PBS; room temperature for 1 hour each step) and incubated overnight at 37° C. in a permeabilization solution (0.01M PBS with 0.2% Triton X-100 and 20% DMSO). Sections were blocked overnight at 37° C. in 0.01M PBS, 0.2% Triton X-100, 6% normal goat serum (NGS), and 10% DMSO. Sections were then incubated in primary antibody (rabbit polyclonal anti-V5 tag; 1:500) in 0.01M PBS, 0.2% Triton X-100, 3% NGS, 5% DMSO, and 10 μg/ml heparin for 72 hours at 37° C. The primary antibody solution was replaced every 24 hours (same 1:500 primary antibody concentration). After overnight washing in 0.01M PBS with 0.2% Triton X-100, sections were incubated in fluorescent-conjugated secondary antibody (Alexa Fluor 647 goat anti-rabbit; 1:500) and a fluorescent Nissl stain (NeuroTrace blue; 1:300) in 0.01M PBS, 0.2% Triton X-100, 3% NGS, and 10 μg/ml heparin for 72 hours at 37° C. The secondary antibody and NeuroTrace solution were replaced every 24 hours (same 1:500 secondary antibody and 1:300 NeuroTrace concentrations). Following overnight washing in 0.01M PBS with 0.2% Triton X-100 and 10 μg/ml heparin, sections were dehydrated with increasing MeOH washes (20%, 40%, 60%, 80%, 100%; room temperature for 1 hour at each step) for tissue clearing. Sections were incubated overnight in 66% dichloromethane (DCM) and 33% MeOH at room temperature followed by two 15-minute washes in 100% DCM. Sections were cleared by incubating in dibenzylether (DBE) for at least 2 hours. Cleared tissue was mounted in DBE on glass microscope slides. Sections in DBE were covered with glass coverslips with silicone spacers and edges were sealed with silicone.

Following curing of the silicone seal overnight, slides were imaged using an Andor DragonFly spinning disk confocal equipped with low-power (2× and 10×) and high-power silicone immersion objectives (30×, 40×, 60×, and 100×) from Olympus. For both low- and high-power imaging, individual image tiles were stitched into composite images with Imaris Stitcher.

Quantification and Statistical Analysis

Quantification of In Vitro Optimization of MORF Expression

For transient transfection experiments, one-way ANOVA was performed with Tukey's post hoc test for significance set at p<0.05. For comparison of transgene brightness between MORF1, MORF2, and MORF3, three random fields from three different mice were imaged in the dorsal striatum of Drd2-Cre/MORF mice. Fields were imaged using standardized exposure for comparison. Statistical analysis used one-way ANOVA with Tukey's post hoc test with statistical significance set at p<0.05. SPSS statistical software (IBM) was used for data analysis.

Quantification of MORF Labeling Frequency

To quantify cell labeling efficiency in immunostained tissue sections, three random fields were imaged in the region of interest for each mouse and MORF⁺ cells were counted as a proportion of the targeted cell population to give a percent labeled for each image. Average labeling frequency is reported for each Cre/MORF combination, n=1-4 mice per genotype. For horizontal cell labeling frequency with MORF2 and MORF3, retina sections from two different slides and two different animals (4 slides total) were stained as described above. The total number of V5⁺ cells and calbindin⁺ (known horizontal cell marker; Haverkamp and Wässle, 2000) cells were counted for each slide. The labeling frequency (in percentage) is the total number of V5⁺ cells over the number of calb⁺ cells.

Reconstruction and Morphometric Quantification of MORF-Labeled Cells

Individual neurons were either reconstructed in Imaris or extracted directly to TIFF stacks with custom MATLAB code and reconstructed in neuTube. Neurons in Imaris were reconstructed automatically and then manually edited, while neurons in neuTube were semi-automatically reconstructed. Only neurons with their complete morphologies contained within the section were reconstructed. The medium spiny neurons were reconstructed in neuTube and then quantified in the following manner: the number of dendrites coming directly from the soma were counted, and the longest of them was measured along its path to obtain a “longest path length” feature. The Euclidean distance from the soma to the tip of the longest primary dendrite was also recorded. Finally, the distance units provided by neuTube were converted to μm by imaging a slide with a calibration grid (10 μm ticks) and processing it in an identical manner to the neurons. The conversion factor was determined to be 2.5 “neuTube units” per μm.

For horizontal cells, each retina was examined for reconstructable retinal horizontal cells (no overlap of dendritic fields and/or terminal branches). The reconstructable retinal horizontal cells were isolated into their own Imaris file and were semi-automatically reconstructed in 3D with Imaris Filament Tracer (Bitplane). All 151 reconstructions were further broken down into three major parts: the dendritic field, the main axon, and the terminal arbor. The main axon is defined as the thickest process from the soma that begins at the soma and ends at the earliest (in order of importance): bifurcation of the main axon, clear axonal thickening, or the last ⅓^{rd} of the horizontal cell along the longest axonal path. The terminal arbor consists of all the processes from the point the main axon ends to the tail end of the cell. Additional quantifications, including tortuosity (Stepanyants et al., 2004), dendritic field size (Yoshimatsu et al., 2014), longest axonal path, terminal arborization length sum, secondary process length, center to soma distance, number of offshoots greater than 10 μm, and total offshoot length, were performed in Imaris and ImageJ for each of the 151 reconstructed P5 retinal horizontal cells. Longest axonal path was determined by taking the longest possible path along the main axon from the soma to the tip of the terminal arbor. The terminal arborization length sum was calculated by summing the lengths of all the filaments that comprise the terminal arbor. Secondary process length was found by summing the length of any dendritic projections coming from the soma that are more than 50% longer than the next longest dendritic projection and that is not the main axon. The center to soma distance was calculated by measuring distance from the middle of the retina to the center of the soma in Imaris. The number of offshoots was found by counting all the processes coming off the main axon that were greater than 10 μm. These were also summed to obtain the total offshoot length parameter.

Clustering Analysis of Cells

Eight cell shape measures (tortuosity, dendritic field size, longest axonal path, terminal arborization length sum, secondary process length, center to soma distance, number of offshoots greater than 10 μm, and total offshoot length) were used for cell clustering. The measures were first clustered using average linkage hierarchical clustering with biweight midcorrelation (a robust correlation; Wilcox, 2011) as measure of similarity, and measures with robust correlations higher than 0.5 were merged and summarized using the first principal component. Specifically, total offshoot length and number of offshoots larger than 10 μm were merged, as were terminal arborization length sum and longest axonal path. All 4 remaining shape measures as well as the two principal components were scaled to mean zero and length 1 and then collected into a single matrix A with 6 columns. Cells were then clustered using average linkage hierarchical clustering of Euclidean distance of rows of the matrix A. Cell clusters were defined from the resulting clustering tree using Dynamic Tree Cut (Langfelder et al., 2008) with arguments deepSplit=1 and minimum cluster size of 5. For cluster visualization, we used t-Distributed Stochastic Neighbor Embedding (t-SNE; Maaten and Hinton, 2008) initialized using the first two singular vectors of A. The clustering analysis was carried out in R.

Results

In Vitro Optimization of Mononucleotide Repeat Types and Context to Improve MORF Reporter Expression Levels.

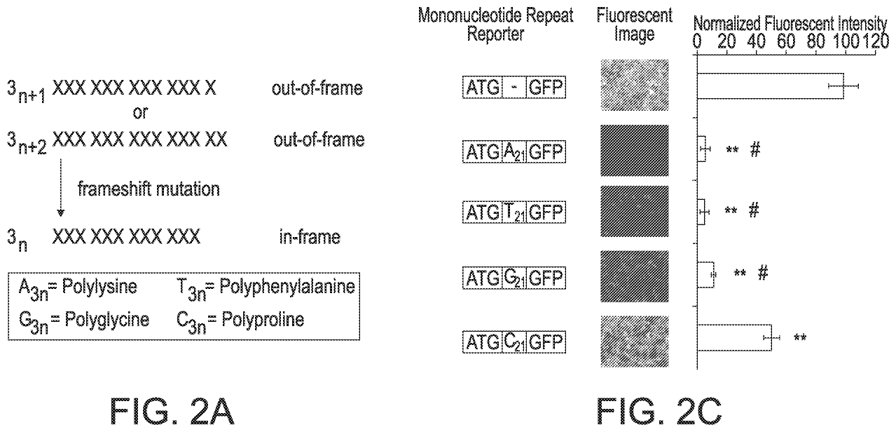

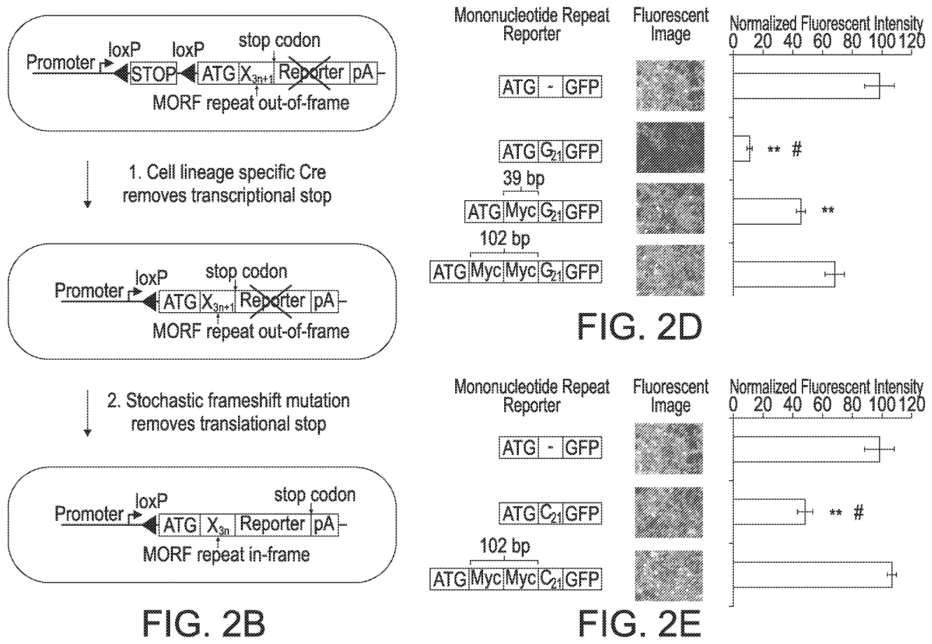

In the current study, we aimed to test whether the MORF-based technology can be optimized to develop mouse lines that confer generalizable, non-invasive, and Cre-dependent sparse and stochastic labeling of brain cells at a frequency that is analogous to Golgi staining (i.e. 1-5% of cells). The general strategy is to integrate MORF reporters into two murine genomic loci (Rosa26 and TIGRE) that support ubiquitous transgene expression and are suitable for Cre-driven reporter gene expression (Soriano, 1999; Madisen et al., 2010; Daigle et al., 2018). In this design, a strong, ubiquitous or tTA-dependent promoter is placed in front of a transcriptional STOP cassette flanked by two loxP sites, which is followed by a MORF reporter cassette consisting of a translational start site (ATG), the mononucleotide repeat itself (FIG. 2A), and a membrane-bound (i.e. farnesylated) reporter and polyadenylation sequence (FIG. 2B). In Cre⁺ cell progenies, the floxed transcriptional STOP sequence is removed, but most cells are still unable to translate the MORF reporter protein due to the mononucleotide repeat acting as an out-of-frame “translational switch”. Only in a small and random subset of Cre-defined cells in which the mononucleotide repeat had undergone stochastic frameshift to an in-frame repeat length (i.e. X₃ₙ₊₁ to X₃ₙ, FIG. 2A), the membrane-bound reporter protein will be translated to label the cellular morphology (FIG. 2B).

To optimize the MORF reporter construct design before generating the reporter mice, we examined several factors including the types of mononucleotide repeats (FIG. 2A) as well as the distance between the ATG start site and the mononucleotide repeat. We tested in vitro the effects of different mononucleotide types on the expression of a C-terminal GFP and found the highest normalized GFP signals from the C₂₁ MORF construct, followed by much lower levels of expression from G₂₁, A₂₁, and T₂₁ constructs (FIG. 2C). Since none of the repeat constructs reached the baseline GFP control signals, we tested whether the juxtaposition of the mononucleotide repeat to the translational start site could lower the reporter protein expression, and showed an insertion of 39 or 102 base pairs (i.e. encoding one or two Myc tags) before the repeats could increase GFP expression. Indeed, such spacer sequences lead to significant increase (e.g. 46% and 69%) increase of GFP signals compared to the baseline G₂₁ or C₂₁ constructs (FIGS. 2D, 2E).

The systematic in vitro optimization revealed that an optimal MORF repeat type is polycytosine, and a DNA spacer (e.g. about 100 base pairs) between the translational start site and the mononucleotide repeat can further enhance the reporter protein expression.

MORF1 and TIGRE-MORF Mice Confer Cre-Dependent Sparse and Stochastic Expression of Direct Fluorescent Proteins

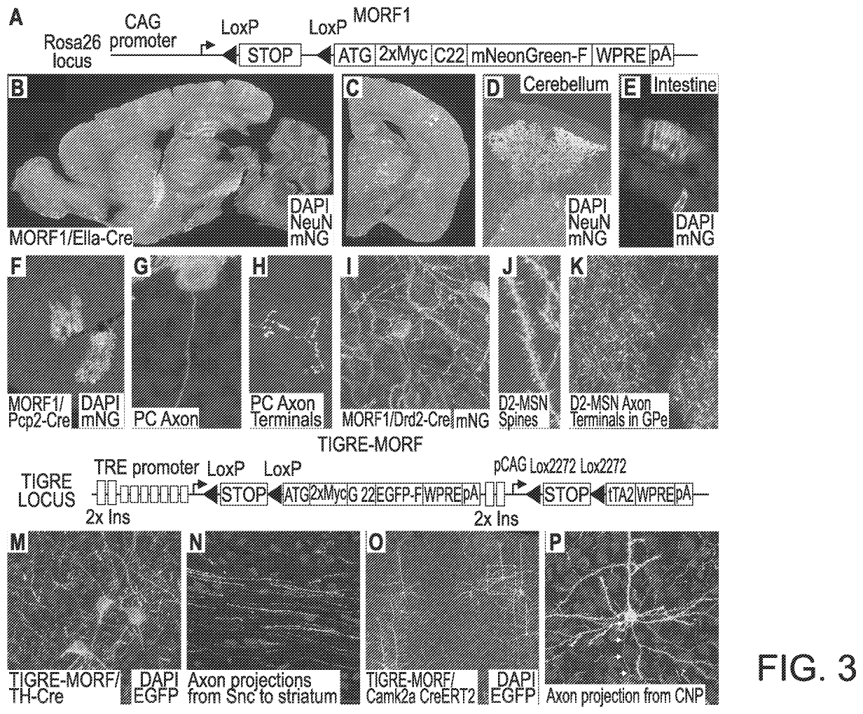

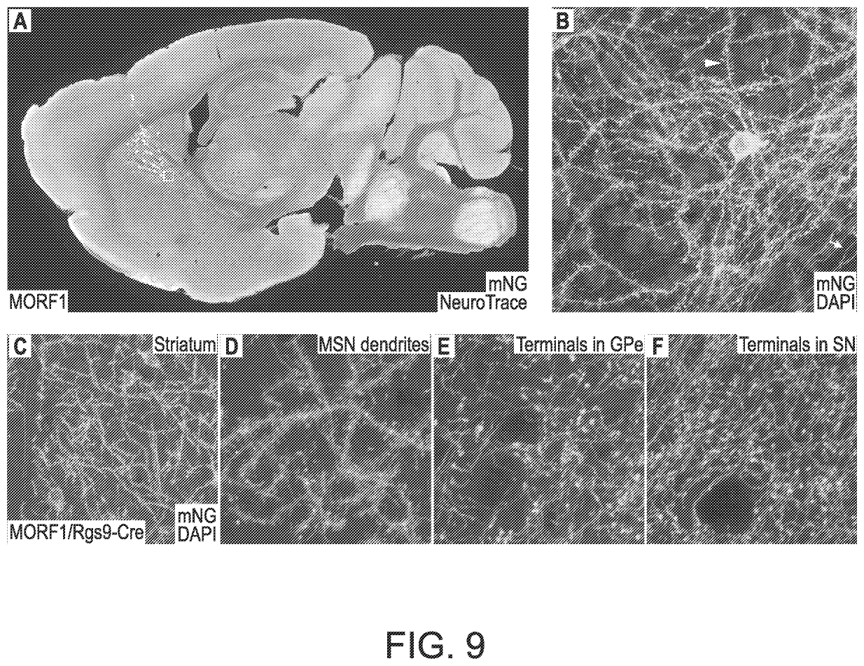

To test if the MORF-based strategy could be used to develop Cre-dependent sparse cell labeling in mice, the MORF1 mouse line was generated by targeted insertion into the Rosa26 locus of a Cre-dependent reporter construct with an optimized C₂₂ repeat and a farnesylated mNeonGreen (mNG-F) reporter, one of the brightest fluorescent proteins (Shaner et al., 2013; FIG. 3A). We confirmed Cre-dependent mNG-F expression by injecting an AAV1-Cre into the striatum of MORF1 mice (FIG. 9A-9B). We next crossed MORF1 to the ubiquitously expressing Ella-Cre mice (Lakso et al., 1996) and used direct fluorescent imaging to confirm the mosaic, sparse and stochastic labeling of neurons and glia throughout the brain (FIG. 3B-3D), and single or a clone of cells in peripheral tissues such as the intestine (FIG. 3E). Moreover, MORF1 can also support Cre-dependent sparse labeling of cerebellar Purkinje cells (PCs) in MORF1/Pcp2-Cre mice (FIG. 3F-3H), striatal D2 medium spiny neurons (D2-MSNs) in MORF1/Drd2-Cre (FIG. 3I-3K), and both D1- and D2-MSNs in MORF/Rgs9-Cre (FIG. 9C-9F). Importantly, the sparsely labeled PCs and MSNs in the MORF1/Cre mice reveal dendrites, dendritic spines, cell bodies, axons, and axonal terminals (FIG. 3, and FIG. 9). Thus, we conclude that MORF1 mice support Cre-dependent sparse and stochastic labeling of certain brain cell populations. One limitation of the MORF1 model, despite using a bright fluorescent protein, is the weak labeling signals for some neuronal populations, such as cortical pyramidal neurons (PN; MORF1/Rbp4-Cre), parvalbumin interneurons (MORF1/Pvalb-Cre), and midbrain dopaminergic neurons (MORF1/TH-Cre). This issue is compounded by the lack of a good mNG antibody for immunostaining.

To enhance the sparse cell labeling signals for a direct fluorescent reporter protein in a Cre-dependent MORF mice, we next employed the TIGRE2.0 transgenic platform (Daigle et al., 2018) using Cre-dependent expression of tTA transcription factor driving a Cre-dependent G₂₂-GFP-F reporter (FIG. 3L). This system allows the transcriptional amplification of Cre-dependent reporter gene expression, achieving high level expression. To test the utility of this TIGRE-MORF mouse line (also known as Ai166) for sparse labeling of Cre-defined neuronal cells, we crossed the line with TH-Cre and Camk2a-CreERT2 mouse lines, and found sparse and very bright labeling of midbrain dopaminergic neurons and their axons (FIGS. 3M and 3N) and cortical PNs, respectively (FIGS. 3O and 3P). Importantly, this mouse line has also been applied to examine the long-range axonal projections of neurons in the cortex and claustrum (Wang et al., 2019). One limitation of TIGRE-MORF mice is the inability to generate live-born double transgenic mice with certain Cre mouse line crosses (e.g. Drd1-Cre, Adora2a-Cre, Rbp4-Cre, Emx1-Cre, and Pvalb-Cre; Table 2), possibly due to high level expression of tTA in the developing embryos (Daigle et al., 2018; Steinmetz et al., 2017), which somewhat limits its general utility.

A Generalizable MORF Mouse Line for Cre-Dependent, Sparse, Stochastic, and High Signal-To-Noise Labelling of CNS Cells

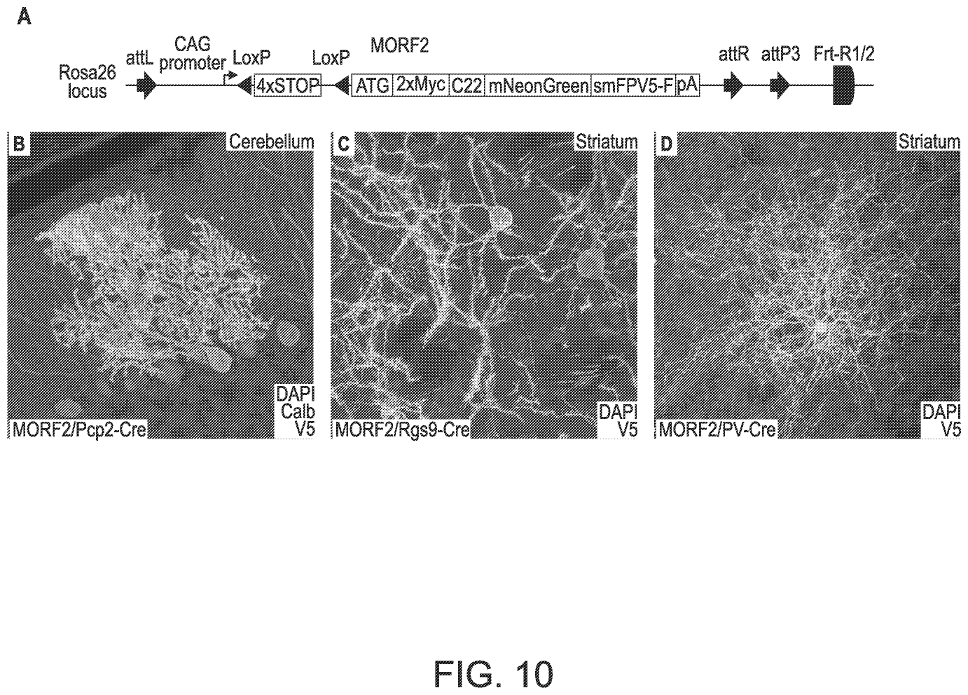

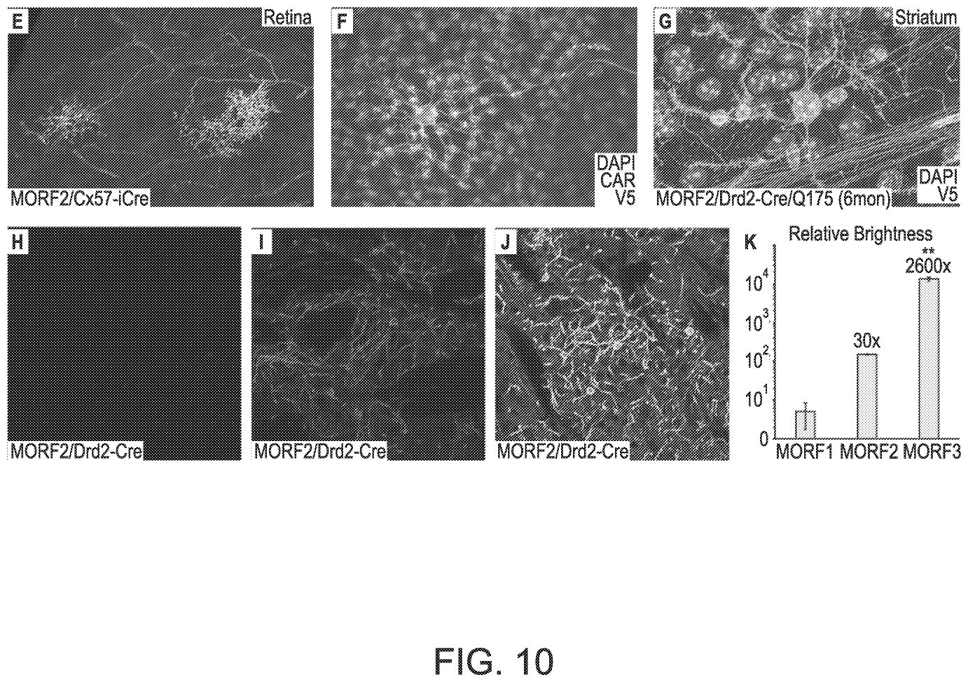

To further generalize the utility of MORF mouse lines, we tested whether “spaghetti monster” fluorescent proteins (smFPs; Viswanathan et al., 2015) could be a superior reporter for MORF-based labeling and imaging of brain cells. SmFPV5 is an immunoreporter with 10 V5 epitope tags embedded in a superfold, non-fluorescent GFP scaffold, and upon anti-V5 staining, it confers high signal-to-noise labeling (Viswanathan et al., 2015). As a proof-of-concept, we first developed MORF2 mice, with a Rosa26 Cre reporter line carrying a C₂₂ repeat and a fusion of mNG and farnesylated smFPV5 (FIG. 2A). We crossed MORF2 with different Cre lines and showed with anti-V5 staining the sparse and cell type-specific labeling of PCs (MORF2/Pcp2-Cre, FIG. 10B), striatal MSNs (MORF2/Rgs9-Cre; FIG. 10C), striatal PV interneurons (MORF2/Pvalb-Cre; FIG. 10D), and retinal horizontal cells (HCs; MORF2/Cx57-iCre; FIG. 10E-10F). The labeled HCs encompasses both the dendrites and axons, suggesting complete labeling of these neurons. Moreover, we showed MORF2 can be used to label D2-MSNs and their dendrites and axons in the striatum of aged (6 m old) Q175 knockin Huntington's disease mouse model (Menalled et al., 2012), and the sparsely labeled D2-MSNs can be co-stained with mHTT aggregates (FIG. 10G and Movie S1). Thus, we conclude that the smFPV5 reporter in MORF2 mice is suitable for long-term labeling and double immunostaining in the mouse brain. However, after our extensive testing of MORF2 mice, we noted in multiple MORF2/Cre crosses (i.e. Drd1-Cre, Drd2-Cre, Cx57-iCre, and Rbp4-Cre) that the offspring were occasionally (<20%) genotyped positive for both MORF2 and Cre but are negative for V5 immunostaining in their brain sections. Such occasional transgene silencing issue had not been found in MORF1 mice, and thus is not caused by the presence of the C₂₂ repeat in the Rosa26 locus. We interpreted that it is likely due to the differences in Rosa26 targeting constructs between these two lines (STAR Methods). Thus, the MORF2 line provides proof-of-concept on the utility of smFPV5 reporters but still has limitations due to occasional transgene silencing.

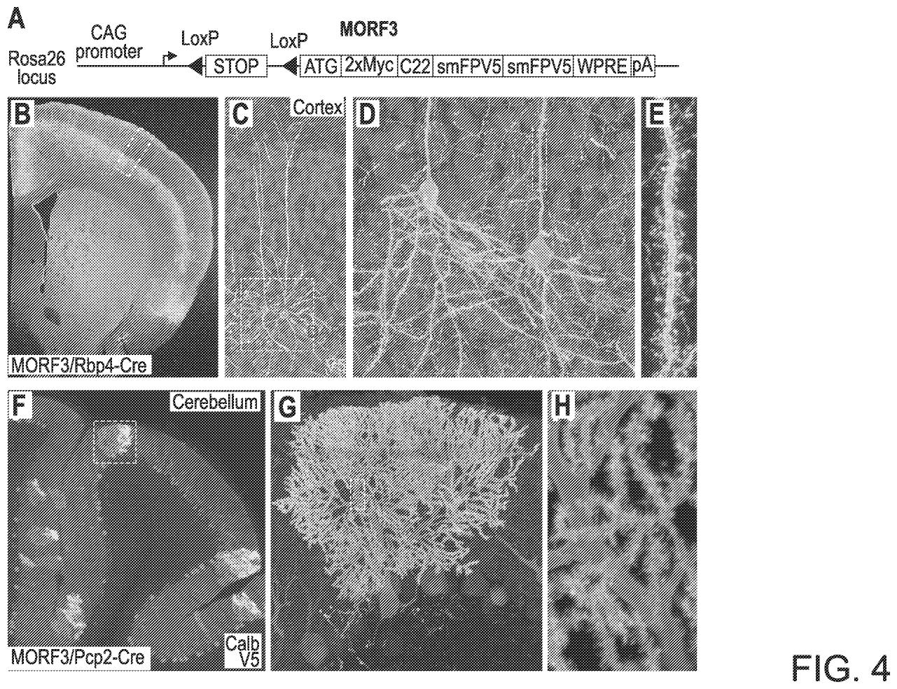

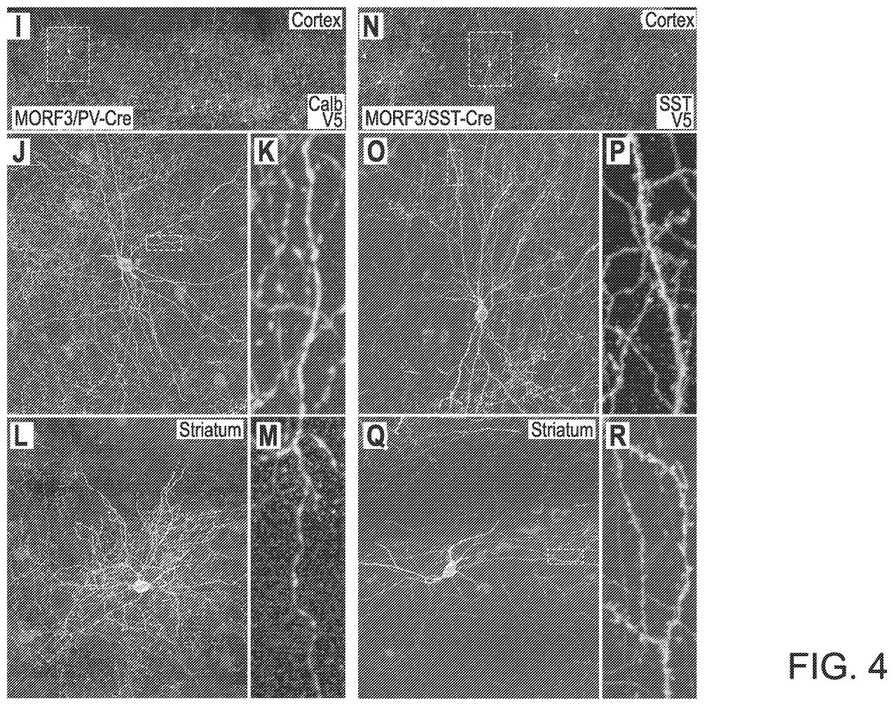

We next sought to develop a truly generalizable MORF mouse line that can be used to cross with any Cre mouse lines and reproducibly confer sparse and stochastic cell labeling, and if possible, further enhance the labeling signal strength of the MORF reporter. To this end, we developed a novel tandem smFPV5 reporter that contains 20 V5 epitope tags followed by a farnesylation signal (td-smFPV5-F; STAR Methods). We used this reporter to generate MORF3 mice based on the MORF1 construct design except substituting mNG-F with td-smFPV5-F (FIG. 4A). We next extensively tested the general utility of MORF3 mice by crossing it with multiple Cre mouse lines and tested a large number of offspring (Table 1; Table 3).

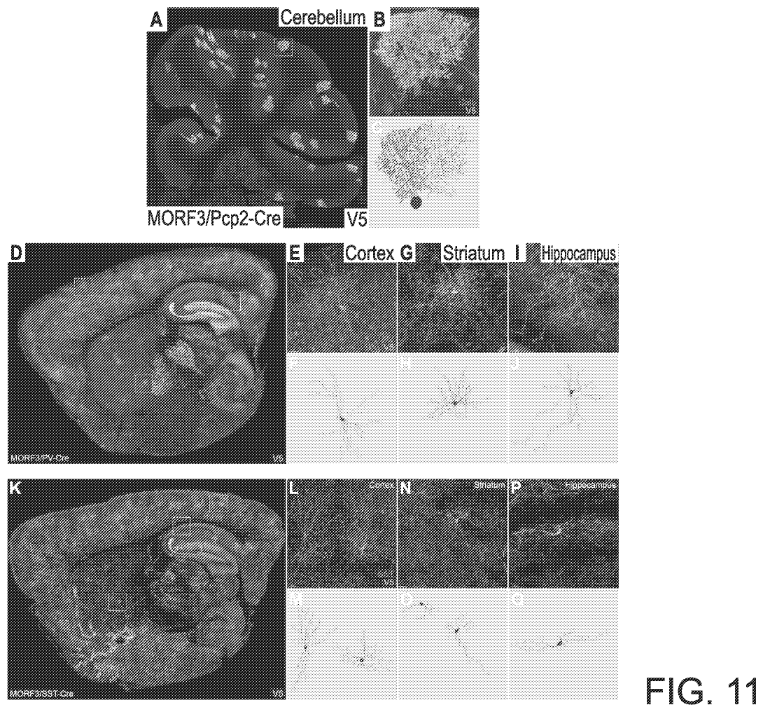

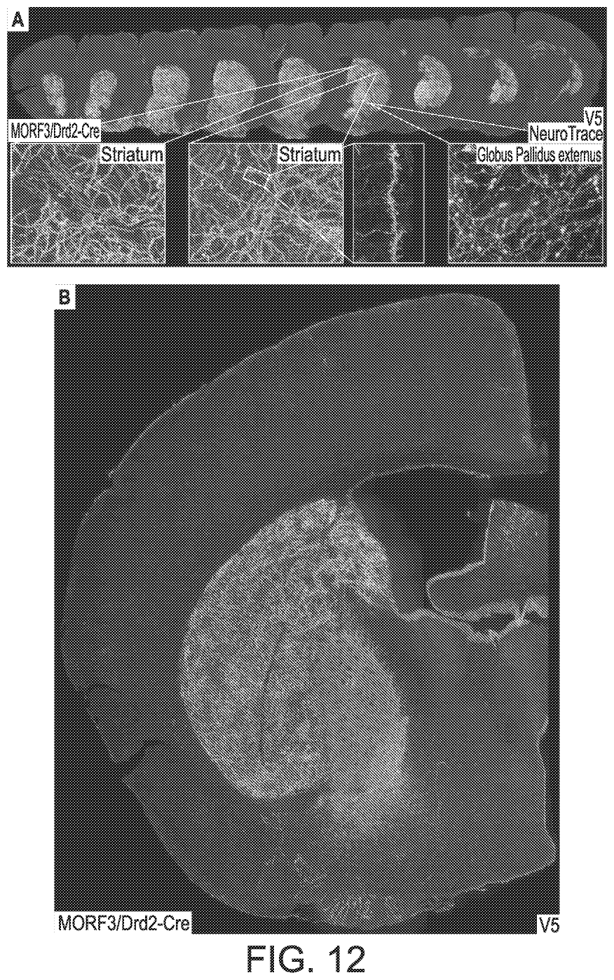

We first tested MORF3 mice for the labeling of long-range projection neurons. To label the layer 5 cortical PNs, we first crossed MORF3 with the Rbp4-Cre mouse and performed V5 immunostaining using brain sections from 8 MORF3/Rbp4-Cre mice (Table 3). We consistently detected sparse and stochastic labeling of L5 cortical PNs and hippocampal neurons (FIG. 4B-4E). The labeling signal is extremely bright to reveal both apical and basal dendritic arbors, dendritic spines, and diffuse axonal projections (FIG. 4B-4E). We next showed MORF3/Pcp2-Cre mice (N=2) label a random subset of PCs, revealing their dendrites and dendritic spines (FIG. 4F-4H; FIG. 11A). These labeled PCs can be readily reconstructed digitally (FIG. 11A-11C). Finally, we crossed MORF3 with D2-Cre and obtained 22 adult double transgenic mice (Table 3), which all showed the sparse labeling of D2-MSNs including their dendrites, dendritic spines, axons, and axonal terminals in the globus pallidus (FIGS. 12A and 12B). Thus, MORF3 mice can confer sparse and stochastic labeling of the long-range projection neurons in the brain, and the labeling appeared complete as they include dendrites, dendritic spines, axons, and axonal terminals.

We next examined whether MORF3 can be used to visualize the morphology of GABAergic interneurons. Multiple Cre mouse lines have been developed for different interneuron classes, but the classical Cre reporters or viral-based labeling often only reveal the dendrites and cannot clearly reveal the densely intermingled and thin axons (Taniguchi et al., 2011). We next crossed MORF3 with parvalbumin (Pvalb) and somatostatin (SST) Cre mouse lines to visualize these two classes of interneurons (Taniguchi et al., 2011). MORF3/Pvalb-Cre mice show sparse labeling of the individual Pvalb⁺ interneurons brainwide, including, but not limited to, the neocortex and hippocampus (FIG. 4I-4M and FIG. 11D-11J). All the MORF3-labeled Pvalb⁺ interneurons display not only the thick dendrites, but also the thin beaded axons (FIG. 4K, 4M). Similarly, MORF3/SST-Cre also sparsely labels individual SST⁺ interneurons and their dendritic and axonal processes brainwide (e.g. cortex, striatum, and hippocampus; FIG. 4N-4R; FIG. 11K-11Q). The neurons were imaged at a resolution of 0.2 μm×0.2 μm×1.0 μm, which permits ready reconstruction of the neuronal dendrites (FIG. 11). Importantly, our preliminary analysis already revealed the diversity of SST and Pvalb interneuron morphologies, supporting the presence of more diverse cell types within these two broad interneuron classes (Huang and Paul, 2019).

Next, we estimated the relative labeling strength of MORF1, MORF2, and MORF3 by crossing all three lines to D2-Cre (Figure S2H-S2K). We found that MORF1/D2-Cre, with direct fluorescent imaging of mNG, has the weakest labeling strength. On the other hand, MORF2/D2-Cre is about 30× and MORF3/D2-Cre 2600× the signal strength compared to MORF1/D2-Cre. Thus, with this example and other evidence, we concluded that MORF3 is superior in terms of both the labeling strength (i.e. signal-to-noise) and generalizability compared to the other MORF lines.

MORF3 Labeling of CNS Microglia and Astrocytes to Reveal their Complete Morphologies

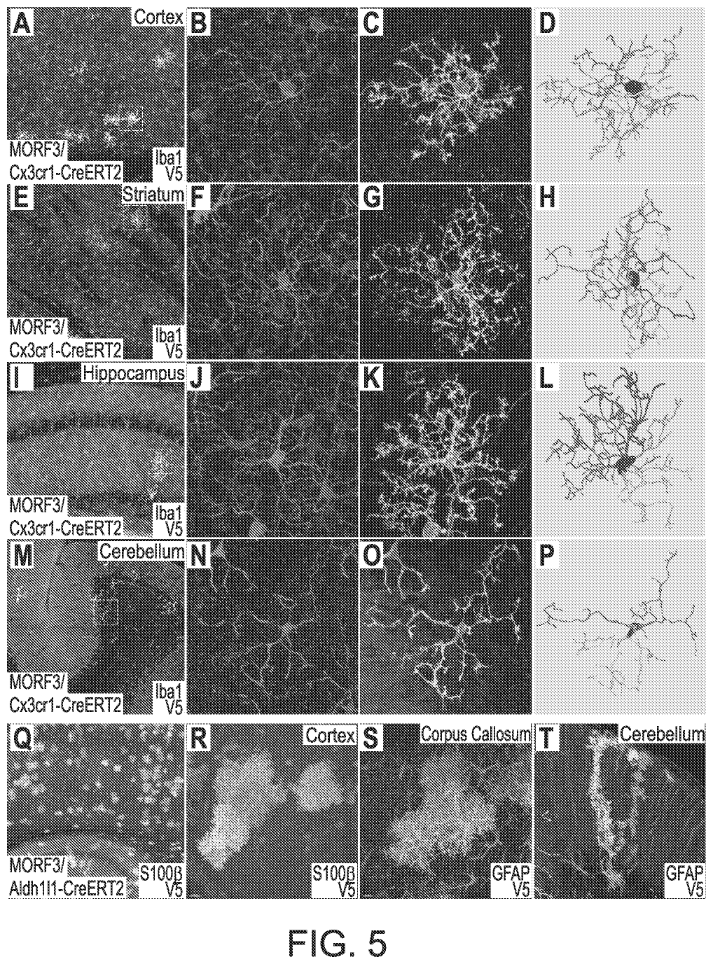

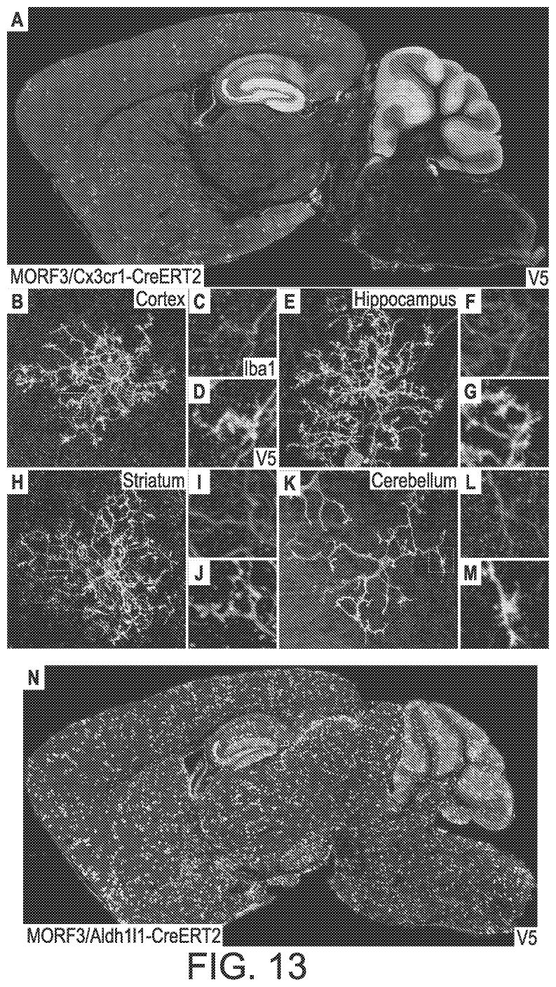

In the mammalian brain, glial cells such as microglia, astrocytes, and oligodendrocytes play key roles in the maintenance and execution of normal brain function as well as in response to various disease processes (Chung et al., 2015; Long and Holtzman, 2019; Khakh et al., 2017). To image the microglial morphology, we used adult MORF3/Cx3cr1-CreERT2 double transgenic mice (N=7; Table S2) with full tamoxifen induction, which normally labels about >80% of the microglia in the brain (Parkhurst et al., 2013). As expected, MORF3/Cx3cr1-CreERT2 mice stochastically labeled about 3.5% of Iba1⁺ microglia brainwide (FIG. 5; FIG. 13A). Unlike Iba1 labeling that only shows the cell body and thick proximal processes, the td-smFPV5-F reporter in MORF3 brightly labeled both the proximal and distal processes of the microglia, including their membranous distal filopodia (FIG. 13B-13M). The labeled microglia can readily be reconstructed using current programs (FIG. 5).

Similarly, crossing MORF3 with Aldh1I1-CreERT2 and giving full tamoxifen induction (see STAR Methods) resulted in much sparser labeling of the astrocyte populations than previously reported with conventional Cre reporter mice (Srinivasan et al., 2016; FIG. 5Q-5T; FIG. 13N). The MORF3 reporter was able to label the membranous branches of astrocytes across multiple brain regions, including the distinct columnar shaped cerebellar Bergmann glia (FIG. 5Q-5T).

Determining the Labeling Frequencies of the MORF Mouse Lines

We have crossed our four MORF mouse lines to 19 different Cre lines and generated a total of 184 double transgenic mice to assess Cre-dependent sparse brain cell labeling (Table 1; Table 3). Importantly, we have analyzed 129 MORF3/Cre mice crossed with 14 different Cre lines, which consistently showed Cre-dependent, sparse, stochastic, and very bright labeling in all the double transgenic mice (Table 1; Table 3). We next assessed the labeling frequency of the MORF mice, which is defined by the percentage of Cre cell progenies that are labeled by the four different MORF/Cre mouse crosses. We estimated the labeling frequency using only the MORF/Cre crosses for cell classes that have readily detectable markers. As shown in Table 1, for 11 different Cre mouse lines, the labeling frequencies of four MORF mouse lines for different classes of neurons and glia range between 1.0% and 5.2%. Overall, the labeling frequency of all the MORF mice has an average of about 3.0%. For the most generalizable MORF3 line, we used eight different Cre lines to show labeling frequencies between 1.2% and 5.2%, and an overall labeling frequency of about 2.4%. Such sparseness is comparable to that of the classical Golgi staining (i.e. 1-5% of brain cells; Luo, 2007), except MORF has the advantage of labeling genetically-defined cell types and is compatible with co-staining of other molecular markers.

MORF3 Mice Support the Immuno-Electron Microscopy Study of Neuronal Ultrastructure

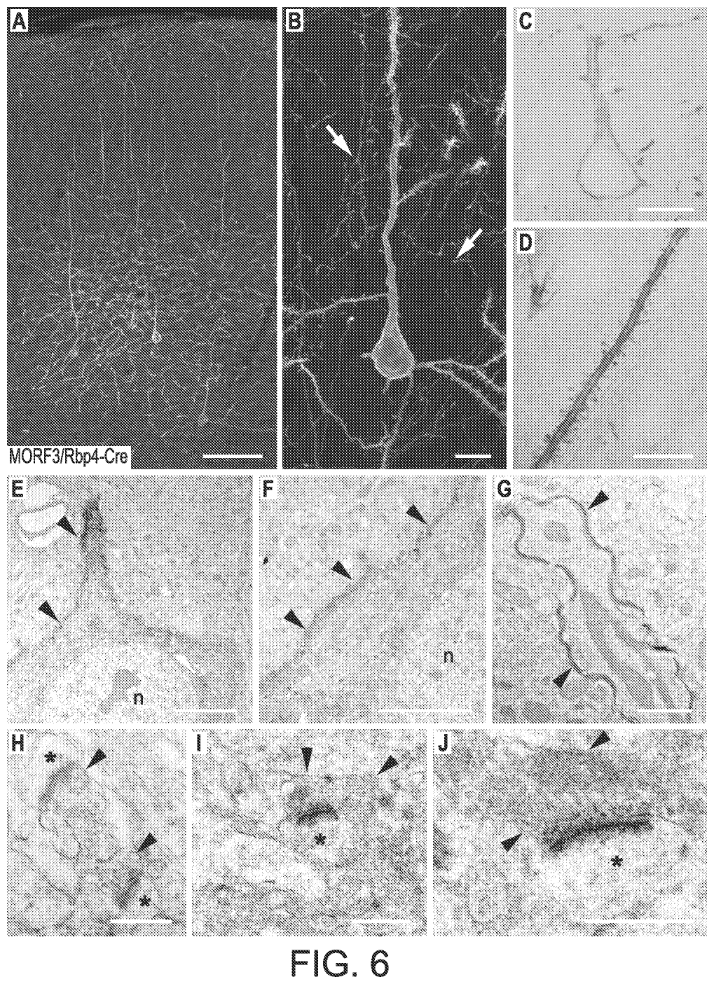

One important goal for studying neurons within a neural circuit is the challenge of imaging their morphology at multiple scales, from the mesoscale dendrites and axons to the nanoscale ultrastructures, including synapses and organelles (Lichtman and Denk, 2011; Zingg et al., 2014; Hintiryan et al., 2016; Oh et al., 2014; Kasthuri et al., 2015; Helmstaedter et al., 2013). Currently there is a gap in neurotechnology that allow the study of genetically-defined neurons at multiple scales. Since smFPs are compatible with immuno-EM (Viswanathan et al., 2015), we tested to see if the sparsely labeled PNs in MORF3/Rbp4-Cre mice can be analyzed with light and electron microscopy. To image the ultrastructures of MORF3-labeled neurons, we used glutaraldehyde in the perfusion solutions (Svitkina, 2009), and tested multiple glutaraldehyde concentrations (e.g. 0.25% to 2.5%) and found 0.5% glutaraldehyde works well for a balance of preserving ultrastructure and maintaining immunoreactivity. As shown in FIG. 6A-6D, anti-V5 immunostaining reveals sparsely labeled detailed morphology of L5 PNs, including the cell bodies, dendrites, axons, and dendritic spines, under confocal and transillumination light microscopy. Strong DAB immunostaining is seen in the semi-thin sections (FIG. 6C-6D) used to select the regions for ultrastructural evaluation. Moreover, under EM with anti-V5 immunostaining followed by a DAB reaction, with mild osmium counterstaining, we can readily detect plasma membrane-bound anti-V5 immunostaining in the cortical PN cell body and proximal dendrite (FIG. 6E-6F), a thin myelinated axon (FIG. 6G), and presynaptic terminals with synaptic vesicles (FIG. 6H-6J), whereas the nucleus is unstained (FIG. 6E-6F). The immuno-EM (FIG. 6E-J) shown were of different MORF3/Rbp4-Cre expressing neurons than those shown in FIG. 6C-D. In principle, however, it is possible to follow the same neuron from the light to the electron microscopic level. Our results demonstrate the feasibility of using standard light and electron microscopic techniques to visualize MORF3 localization, at both the cellular and ultrastructural levels. For volumetric EM reconstructions, a different technical approach is necessary, such as intracellular labeling of neurons and automated sectioning and reconstruction software (e.g., Baena et al., 2019; Guérin et al., 2019). This provides a way for brainwide imaging of genetically-defined, sparsely labeled neurons at both the mesoscale and nanoscale.

An Imaging Platform to Study the Brainwide Morphology of MORF3-Labeled Cells

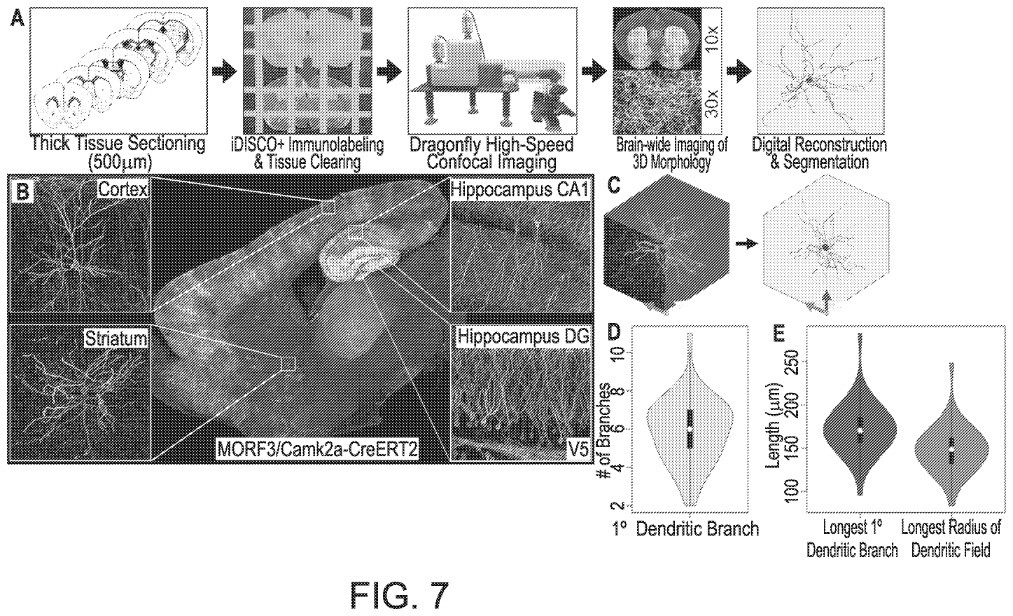

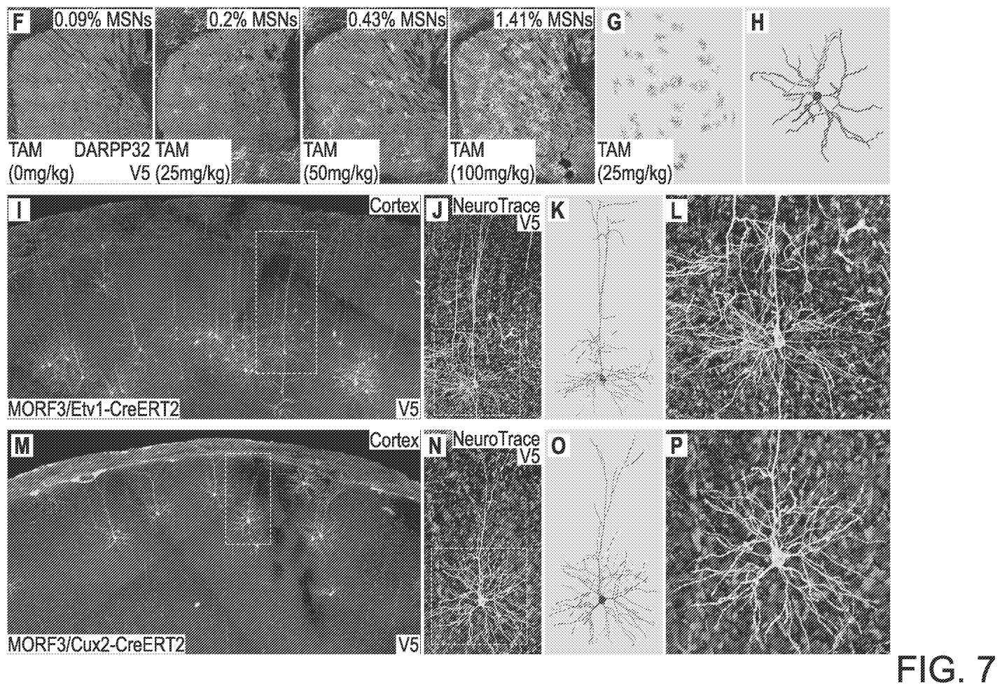

To systematically study the morphology of MORF-labeled neurons and glia throughout the brain, we also need a brain tissue processing and imaging pipeline to capture the intact three-dimensional (3D) cellular processes. To this end, we have developed a pipeline to tissue-clear and image MORF3-labeled neurons or glia in thick-cut (500 μm) serial brain sections (FIG. 7A). To overcome the limitation of light imaging depth in brain tissues due to light-scattering in a lipid-rich brain environment (Heintzmann and Ficz, 2013; Richardson and Lichtman, 2015), and the limitation of antibody penetration with passive immunostaining of the td-smFPV5-F reporter, we adapted the iDISCO+ tissue-clearing protocol (Renier et al., 2014; 2016) to our MORF3/Cre thick brain sections (see STAR Methods). In addition, we counterstained the brain sections with NeuroTrace (fluorescent Nissi) to enable the registration of each 10× imaged serial brain section onto a digital reference brain atlas, such as the Allen Reference Atlas (ARA, Dong, 2008).

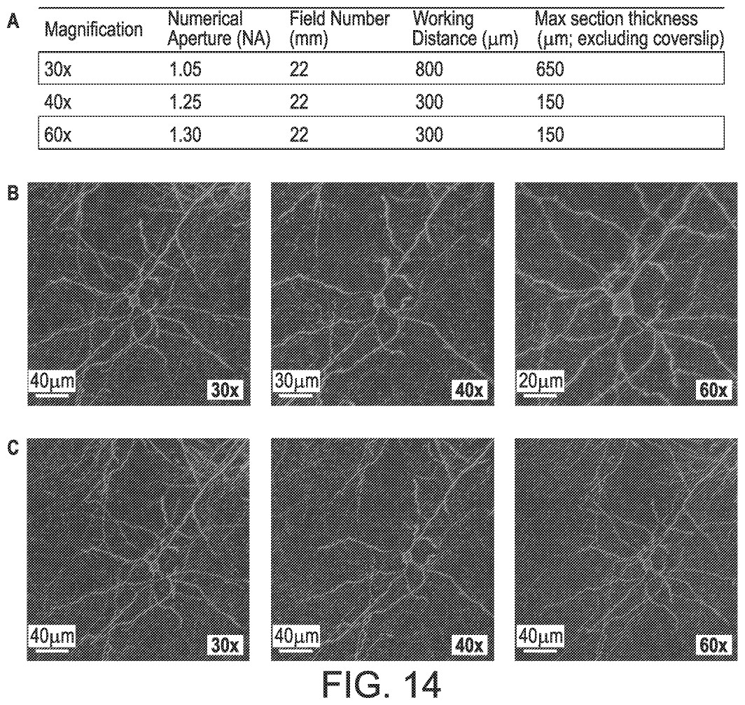

In order to perform high-resolution, brainwide imaging of thick MORF3/Cre-labeled brain sections, we utilized a DragonFly confocal microscope (Andor, Oxford Instruments) that confers faster imaging times compared to a conventional laser-scanning confocal, and an Olympus microscope with high-power silicone immersion objectives that provide long working distances (up to 800 μm) with high numerical apertures (FIG. 14). Although the 40× and 60× objectives have higher resolving power, they are limited to a working distance of 300 μm. The 30× objective has a superior working distance of 800 μm to enable imaging of the entire depth of the 500 μm thick brain sections and can resolve the major dendritic branches comparable to 60× imaging (FIG. 14). As an example of our tissue processing and imaging pipeline, FIG. 12A shows a complete 10× image series of 500-μm thick brain sections of a MORF3/D2-Cre mouse brain, and a 30× high-resolution image of one of these sections is shown in FIG. 12B. Moreover, selected regions in these brain sections re-imaged at 100× reveal that the dendritic spines in the striatum and the axonal terminals in the globus lallidus externus (GPe) are intact and can be readily imaged, if needed (FIG. 12A).

Ultra-Sparse Labeling of Densely Packed Neurons to Facilitate Digital Reconstruction

One rate limiting factor in the morphological analysis of MORF-labeled neurons is the ability to digitally reconstruct the morphology of all the sparsely labeled and imaged brain cells. With the Golgi-like labeling frequency of 1-5%, MORF3 labeling of certain brain cell populations (e.g. PCs, PV⁺ and SST⁺ interneurons, and microglia) that are relatively well separated from each other can be readily reconstructed with current programs (Peng et al., 2010; Li et al., 2019). However, for the projection neurons of the cortex and striatum, the MORF3/Cre mice (FIG. 4; FIG. 12) with labeling frequency of 1-5% still appear too dense for reconstruction with currently available programs. Thus, we reasoned that for the neuronal populations with relatively dense packing and intermingling of their processes, a further reduction in labeling frequency will be necessary to facilitate their morphological reconstructions.

To test such an idea, we combined MORF mice with well-characterized inducible Cre mouse lines and reduced the labeling frequency well below 1% by fine-tuning the Cre activity (Madisen et al., 2010; Wang et al., 2019). As a proof-of-concept, we first crossed MORF3 with a leaky inducible Cre mouse line, Camk2a-CreERT2 (Madisen et al., 2010). Indeed, we can achieve the ultra-sparse labeling of both PNs and MSNs in the MORF3/Camk2a-CreERT2 brains without CreER induction (FIG. 7B; Movies S2 and S3). The MSN labeling in these brains is extremely sparse (0.09%; FIG. 7F) and their dendritic arbors can be readily reconstructed. Our analysis of 40 reconstructed MSNs imaged from 500-μm thick brain sections from the MORF3/Camk2a-CreERT2 brains reveal the Euclidean distance of the tip of the longest dendritic branch to the soma (i.e. the radius of the dendritic field) has a median of just below 150 μm and can be as long as 250 μm (FIG. 7D, 7E). Therefore, to encompass the entirety of the 300-400 μm dendritic field of an average MSN, it is necessary to image brain sections of at least 400 μm in thickness. This represents a significant advance in the study of full dendritic morphology of MSNs, since the prior MSN morphological studies often used brain sections with a thickness of 250 μm or thinner (NeuorMorpho.org, Ascorli et al., 2007) and therefore likely contained partial dendritic processes of these neurons.

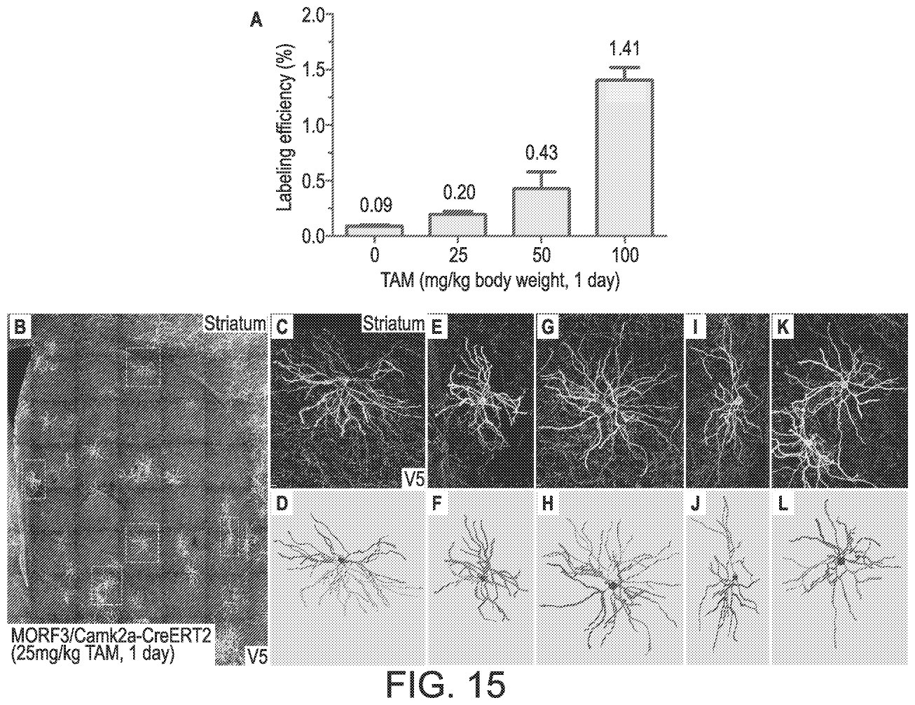

We next tested the idea that the MSN labeling density in the MORF3/Camk2a-CreERT2 brains can be gradually increased with escalating tamoxifen induction, so that we can achieve a higher labeling density and all the labeled MSNs can still be digitally reconstructed. Indeed, with one-day tamoxifen inductions of 25, 50 or 100 mg/kg, the MSN labeling frequencies are 0.2%, 0.43%, and 1.41%, respectively (FIG. 7F; FIG. 15A). Importantly, at the labeling frequency of 0.2% (with about 3000 labeled MSNs per brain), the labeled MSNs can still be readily reconstructed using our current programs (FIG. 7G, 7H; FIG. 15B-15L).

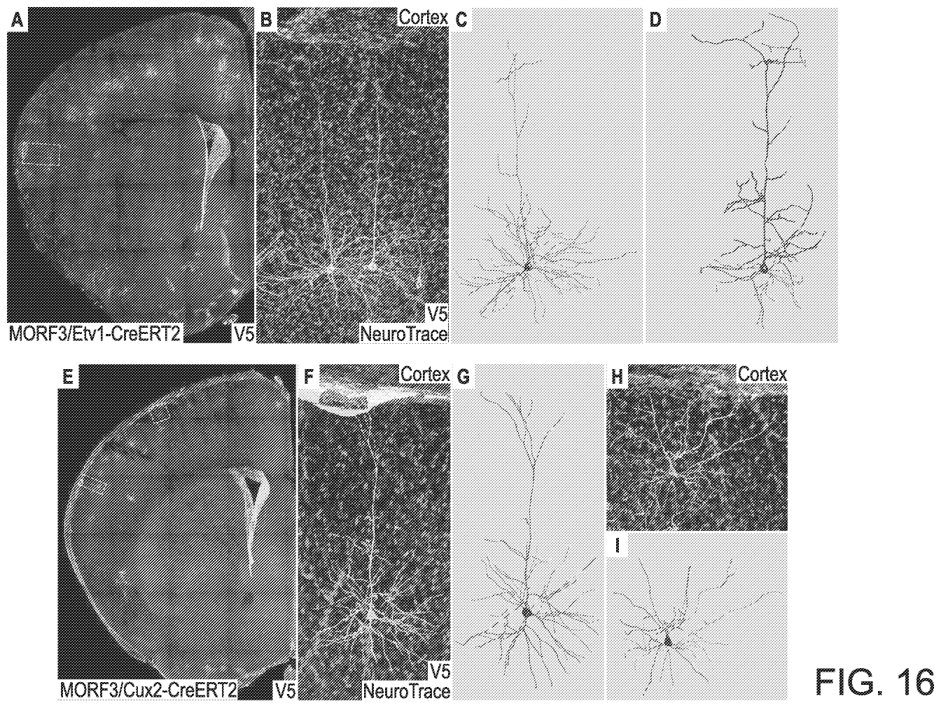

To test whether MORF3 can also ultra-sparsely label cortical PNs for neuronal reconstruction, we crossed MORF3 with Etv1-CreERT2 to label layer 5 PNs and Cux2-CreERT2 to label layer 2-4 PNs (Madisen et al., 2010). With tamoxifen induction, the double MORF3/Etv1-CreERT2 mice label the L5 PNs at a very low density, and the labeled neurons can be readily reconstructed digitally (FIG. 7I-7L; FIG. 16A-16D). Similarly, the MORF3/Cux2-CreERT2 mice can readily label the L2-L4 cortical PNs at an ultra-sparse density, and the labeled cortical PNs can also be readily reconstructed with our current pipeline (FIG. 7M-7P; FIG. 16E-16I).

Applying MORF3 Mice to Study Axonal Development of Retinal Horizontal Cells

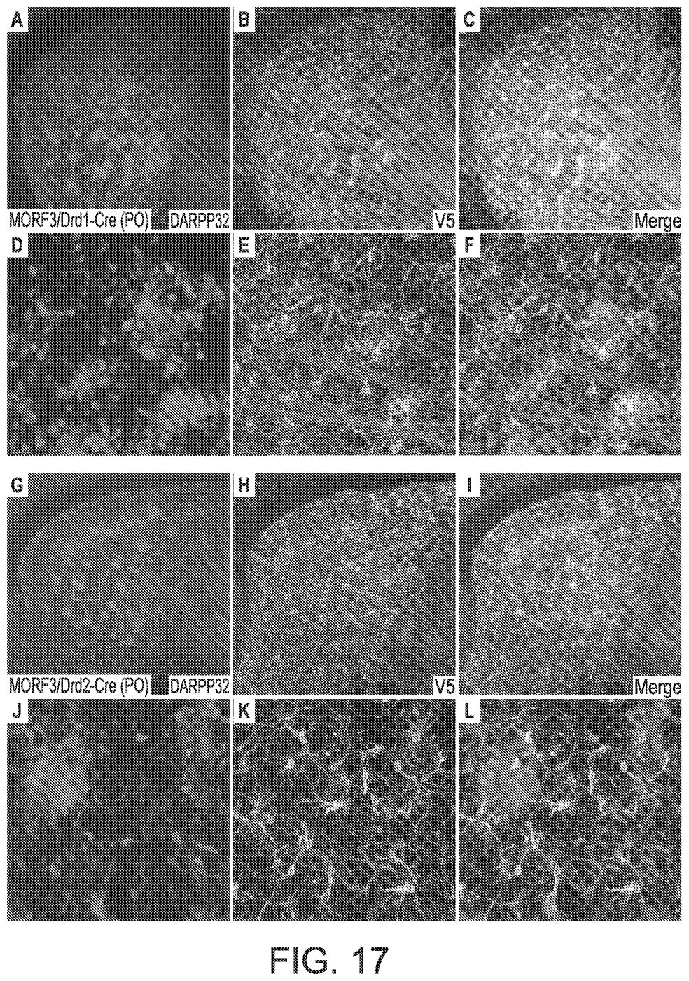

One distinct advantage of MORF3-based genetic sparse cell labeling is to study the morphology of developing neurons in embryos or early postnatal days when other sparse labeling methods, such as viral-based labeling or slice microinjections, are not feasible. As a proof-of-concept, we first tested whether MORF3 can be crossed to D1-Cre and D2-Cre lines to label D1-MSNs and D2-MSNs at postnatal day 0 (P0). As shown in FIG. 17, we obtained MORF3/D1-Cre and MORF3/D2-Cre mice (N=2 per genotype), and immunostained for both V5 epitope and DARPP-32, an early MSN differentiation marker. We found consistent sparse labeling of the differentiating D1-MSNs and D2-MSNs (FIG. 17) in the two double transgenic mice, revealing their developing dendritic processes (FIG. 17E, 17K). Interestingly, there appears to be more overlap of DARPP-32⁺ striosomal neurons with D1-MSNs (FIG. 17C, 17F) than their overlap with D2-MSNs (FIG. 17I, 17L), a finding consistent with a prior study (Biezonski et al., 2015).

We next sought to provide a proof-of-concept that the MORF mice could be used to study neurodevelopmental questions that were not feasible with prior sparse labeling methods (e.g. viral-based labeling). We focused on examining the morphological development of retinal horizontal cells (HCs), which constitute about 2-3% of the neurons in the murine retina (Jeon et al., 1998; Whitney et al., 2011). They are inhibitory interneurons, and in rodents, they have spherical dendritic trees that synapse with cone photoreceptors, while a long and thin axon with dense axonal terminals that synapse with rod photoreceptors (Peichl and González-Soriano, 1994). The morphology of HCs are important to their functions in local and global visual signal processing in the retina (Masland, 2012; Chapot et al., 2017; Chaya et al., 2017; Ströh et al., 2018; Drinnenberg et al., 2018). In rodents, most HCs become postmitotic by birth, and like other retinal cell types, HC neurogenesis and differentiation follow a center to peripheral gradient in the retina (Rapaport et al., 2004; Young, 1985). The development of HCs has been enigmatic due to the unusual soma migration and morphological plasticity in both the dendritic and axonal systems (Poché and Reese, 2009; Boije et al., 2016). Although HC development in different vertebrates has been extensively studied for over half a century (Poché and Reese, 2009; Boije et al., 2016), very little is known about the developmental axonal features of the HCs. The challenge to study the postnatal axonal development of HCs is the difficulty to sparsely and completely label the morphology of early postnatal HCs using existing methods (Peichl and González-Soriano, 1994; Soto et al., 2018; Huckfeldt et al., 2009).

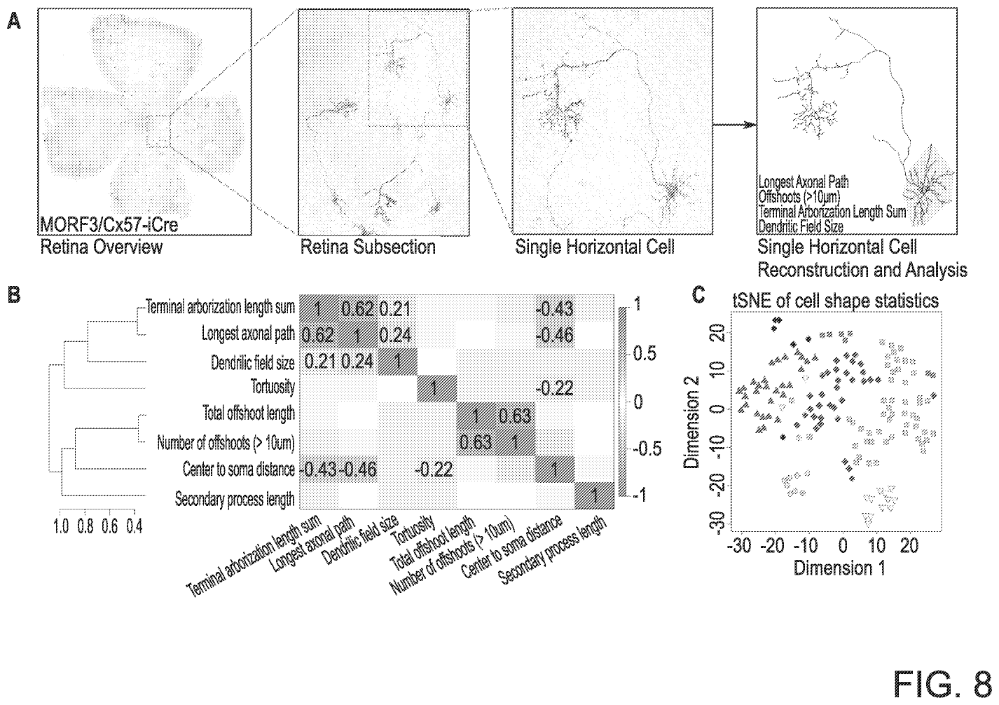

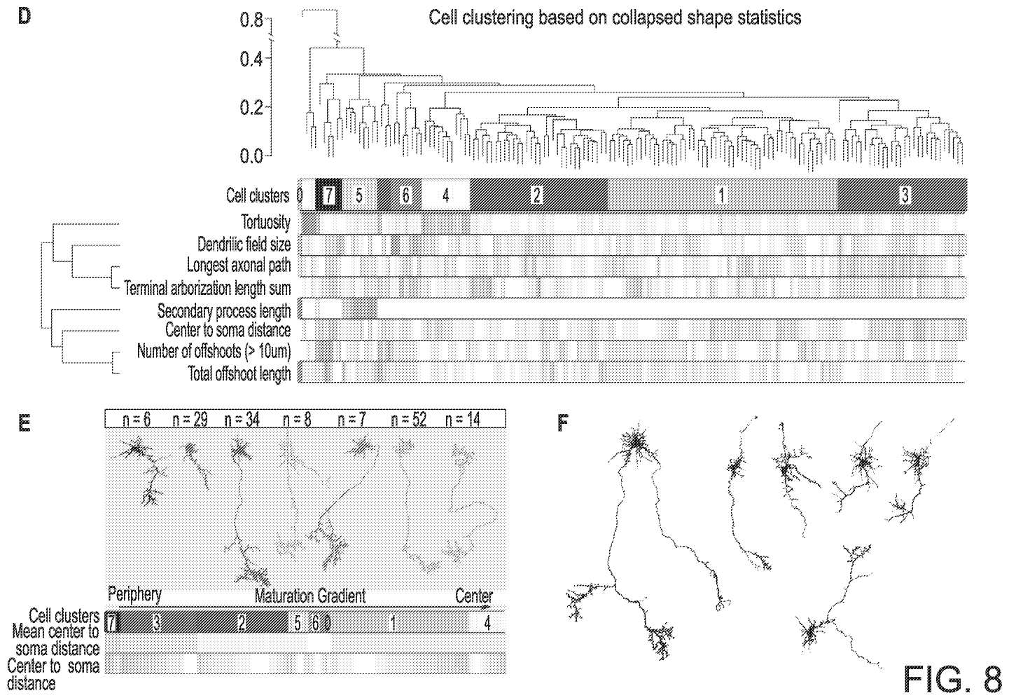

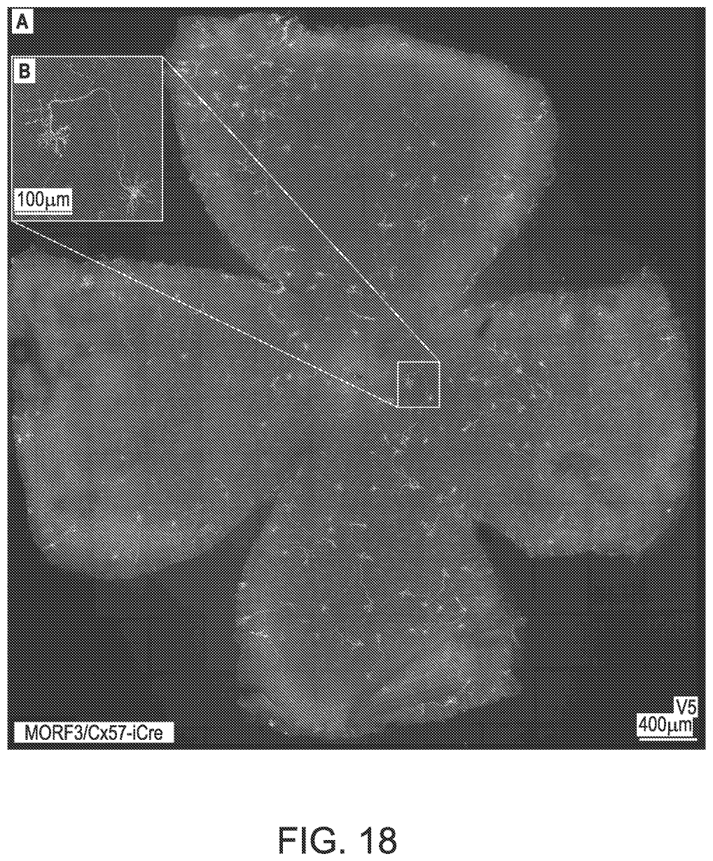

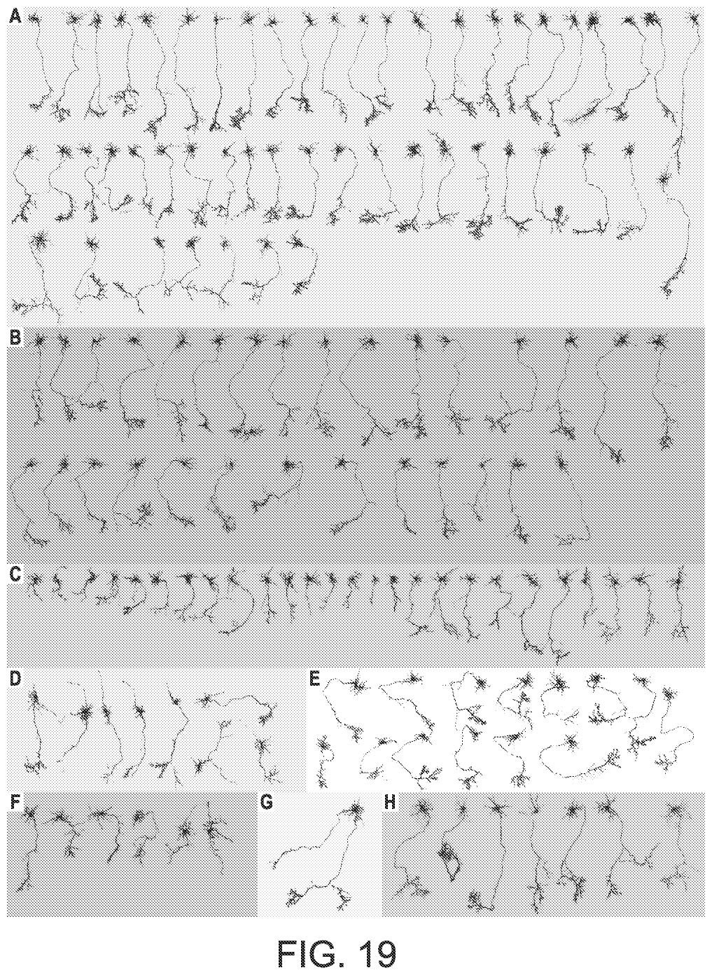

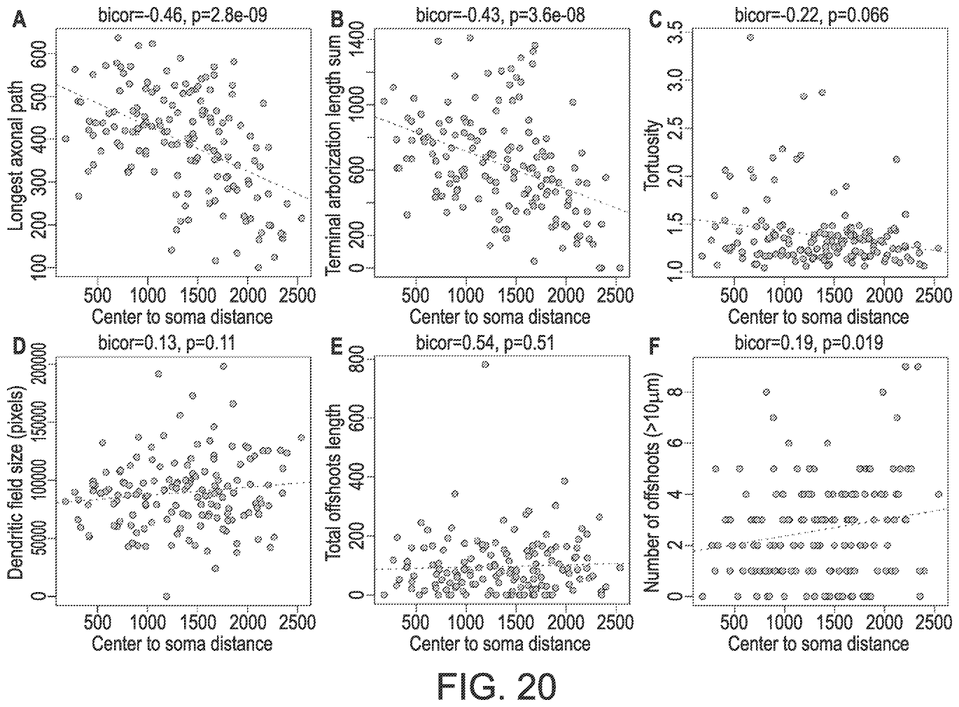

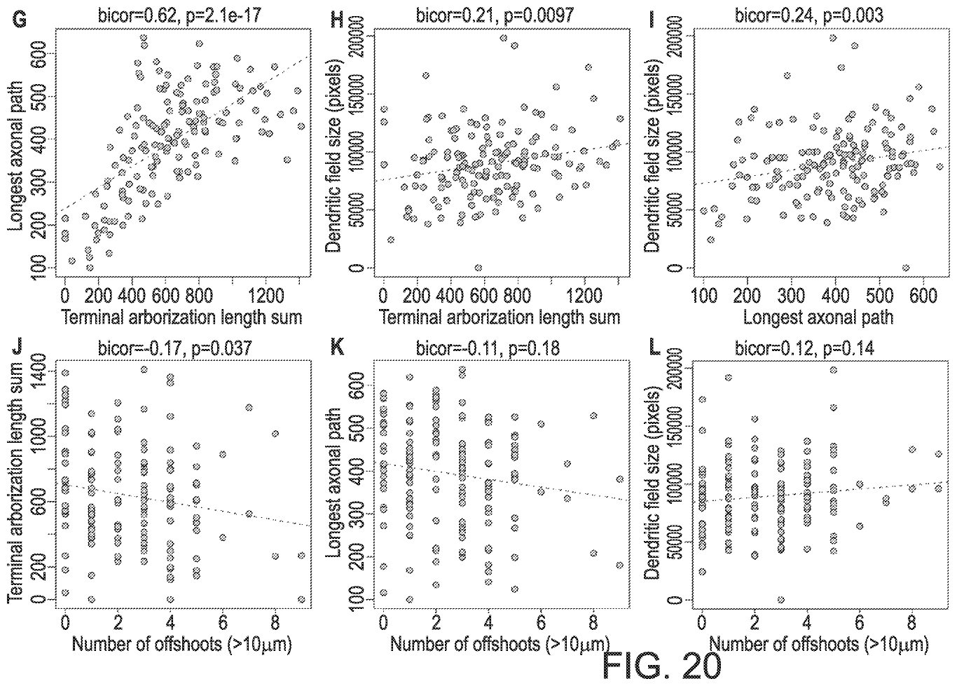

We sparsely labeled developing (postnatal day 5) HCs by crossing MORF3 with Cx57-iCre (Hirano et al., 2016; FIG. 18). We imaged the entire retina at 30× magnification for three different MORF3/Cx57-iCre retinas, with each having about 1.2% of HCs labeled with the td-smFPV5-F reporter (Table 1; FIG. 18). Next, we digitally reconstructed the full morphology of 151 developing P5 HCs (FIG. 19), which allowed us to extract five distinct morphological features: dendritic field size, axonal terminal arborization (length sum), longest axonal path, tortuosity of axons, and distance between the center of the retina to the neuronal soma (i.e. eccentricity; FIG. 8A). Importantly, we also defined novel morphological features observed in the developing HCs, including the length and number of axonal offshoots, which are extra axonal branches along the long thin axon (FIG. 8A), and occasionally second long, axon-like process (FIG. 8B, 8F). Importantly, to our knowledge, this latter rare developing HC subtype (7/151) has never been described before. Correlation analyses and binary scatter plots of all seven morphological features and soma eccentricity provide new insights (FIG. 8B; FIG. 20). Interestingly, the longest axonal path and the terminal axonal arborization are strongly correlated with each other, and they are significantly negatively correlated with soma eccentricity. This finding suggests that the central to peripheral retinal developmental gradient is strongly correlated with the HC axonal maturation at P5 (FIG. 8B, FIG. 20G, 20A, 20B). However, the dendritic field size is only modestly correlated with the two axonal parameters (FIG. 8B, FIG. 20H, 20I), and not significantly correlated with the soma location at all (FIG. 20D). This is consistent with prior findings that independent cues may regulate the maturation of HC dendrites and axons (Poché and Reese, 2009; Soto et al., 2018). Moreover, the presence of the novel axonal offshoots and the length of such offshoots are not correlated with other morphological features or soma eccentricity, suggesting these excessive early axonal growths may be pruned through a process that is not closely linked to the main axonal or dendritic development.

We next performed unbiased clustering analyses using all seven HC morphological features and soma eccentricity and defined seven distinct developing HC clusters (FIG. 8D), which can be visualized in a tSNE plot (FIG. 8C). Importantly, each developing HC cluster has a unique combination of morphological features (FIG. 8D), which can be better visualized when we rank these cell clusters based on their soma eccentricity along the maturation gradient (FIG. 8E). As expected, the Black (Cluster 7) and Brown (Cluster 3) Clusters have the most soma eccentricity and immature morphological features (short axons and smaller dendritic fields), and they separate from each other based on the number of axonal offshoots. The HCs in the Blue (Cluster 2), Red (Cluster 6), and Turquoise (Cluster 1) Clusters appear to all have long thin axons but differ from each other in terms of dendritic field size. The Yellow Cluster (Cluster 4) appears to be the most mature as they are located closest to the center of the retina and have long and tortuous axons (FIG. 8D, 8E). The most peculiar and unexpected cell cluster is the Green Cluster (Cluster 5), which consists of the HCs with two long axon-like processes (FIG. 8D-8F). The somas for the Green Cluster HCs are in the middle of the center-peripheral gradient, suggesting they belong to immature HCs (FIG. 8E, 8F). Since such bi-axonal HCs are missing in the adult retina, and some of the Green Cluster HCs show one axon with more elaborate processes than the other one, it suggests the extra axons could be retracting or these types of HCs are eliminated by the adult stage. In summary, the MORF3/Cx57-iCre mice allowed us to discover novel developmental axonal features of HCs, including unbiased morphology-based HC clusters and novel axonal branching patterns during HC development.

Discussion

Cajal's systematic investigation of the morphology of Golgi-stained neurons in vertebrates laid the foundation for modern neuroscience (Swanson and Lichtman, 2016). Despite a century of progress, current neurotechnology for studying neuronal and glial cell morphology are mostly limited to only dozens of reconstructed cells per study (e.g. Gertler et al., 2008; De Biase et al., 2017), and the few studies with morphological analyses of 1000-2000 neurons are limited to large-scale organizational efforts (Winnubst et al., 2019; Markram et al., 2015). Thus, to systematically study the morphology of genetically-defined neurons and glial cells in the mouse brain, there is a critical need for a simple, generalizable genetic method for sparse, stochastic, and complete labeling of brain cells that can be readily applied to study all different cell types in the brain. Here we describe four MORF mouse lines that have been iteratively optimized for Cre-dependent sparse and complete labeling of 1-5% of Cre-defined cells, a labeling frequency that is akin to the traditional Golgi staining method and hence suitable for brainwide morphological analyses of genetically-defined neurons and glial cells.

Our suite of MORF mice, particularly MORF3 mice, represent a significant technological advance in genetically-directed sparse cell labeling for studying the morphology of neurons and glial cells compared to prior methods. The first is the simplicity of our method for genetic cell labeling by simply crossing the MORF mice with any Cre mouse line, which is much simpler, cheaper, and scalable compared to traditional methods such as microinjections in brain slices and viral-based labeling. The second major advantage demonstrated with MORF3/Cre mice is the scale and distribution of genetically labeled neurons and glia: MORF3/Cre labels about 1-5% of Cre⁺ neurons and glia distributed stochastically brainwide. Based on the labeling frequency (Table 1) and the total number of cortical interneurons and microglia (Ero et al., 2018; Tremblay et al., 2016), MORF3/Pvalb-Cre labels about 27K Pvalb⁺ cortical interneurons, MORF3/SST-Cre labels about 27K SST⁺ cortical interneurons, and MORF3/Cx3cr1-CreERT2 labels about 199K cortical microglia. Moreover, we provide a strategy to use low-level tamoxifen induction of MORF3/CreER mice to label about 3000 MSNs in each MORF3/CamK2-CreERT2 mouse brains and over 1000 cortical L5 and L2-L4 PNs in MORF3/Etv1-CreERT2 and MORF3/Cux2-CreERT2 mice, respectively. In each case, we provide evidence that the labeled neurons or microglia can be digitally reconstructed with current algorithms. Thus, an important advance of MORF-based reporter mice is sparse and stochastic genetic labeling of thousands to tens of thousands of neurons or glial cells per brain and at density and clarity that are amenable to digital morphological reconstructions.

Our method is conceptually distinct and has greatly expanded utility compared to prior Cre-dependent reporter mouse lines for sparse cell labeling. The MADM mice relies on Cre-dependent mitotic recombination of two reporters located on two homologous chromosomes and are limited to Cre lines that express during mitosis, and cannot be used in combination with Cre lines that express in postmitotic neurons or glia cells (Zong et al., 2006). Another mouse line achieves low Cre-dependent labeling by placing long “spacer” DNA (e.g. >10 kb) in between two loxP sites (Ibrahim et al., 2018), but its labeling frequencies still appear too high (8-10%) and reporter (YFP) is suboptimal (Araki et al., 1997). Brainbow mice confers multi-color labeling of axons of Cre-defined neurons, but its dendritic labeling appear too dense to resolve (Livet et al., 2007). Moreover, compared to the exclusive use of CreER inducible lines for sparse cell labeling (Badea et al., 2003; Badea et al., 2009), MORF3 has the versatility to be combined with all the existing Cre mouse lines to obtain Cre-dependent sparse cell labeling. Finally, viral-based sparse neuronal labeling has certain limitations such as invasiveness of the viral studies, poor infectivity of AAV to microglia (Rosario et al., 2016) and the inability for viral-based labeling to study neurons and glia during embryonic or early postnatal development. In contrast, MORF3-based labeling can readily label the intact morphologies of microglia (FIG. 5; FIG. 13) and developing MSNs and HCs (FIG. 8; FIG. 17) for morphological studies. Together, our suite of MORF mice greatly expand our capability for brainwide sparse labeling of genetically-defined neurons and glia for detailed analysis of their morphologies. Moreover, our optimized MORF3 mice, provide the first simple, generalizable, and scalable solution for genetically-directed sparse cell labeling to systematically study brain cell morphology in the mammalian brain.

The MORF mice allow advances in addressing important neurobiological and disease research questions involving the brainwide analyses of genetically-defined neuronal or glial cell morphology. The unbiased analyses of neuronal morphology and integrating such data with molecular phenotyping facilitate more precise brain cell type classification, a major goal of the U.S. BRAIN Initiative Cell Type Classification Consortium (Ecker et al., 2017; Zeng and Sanes, 2017). Another important application of the MORF-based sparse cell labeling is to examine the neuronal and glial cell morphology throughout embryonic and postnatal development, as demonstrated by our proof-of-concept study of axonal development of postnatal retinal HCs Finally, our MORF mice should greatly expand the use of brainwide neuronal and glial cell morphology to more precisely define cellular pathology in vulnerable neuronal or glial cell types in mouse models of brain diseases (Duman and Aghajanian, 2012; Adalbert and Coleman, 2013; Fu et al., 2018; Lee et al., 2018; Wang et al., 2014). With MORF mice labeling thousands or more genetically defined neurons or glial cells per brain and at densities and labeling strength that are suitable for morphological reconstruction, we envision MORF mice could be used for large-scale, brainwide quantitative analyses of neuronal and glial cell pathology, which in turn should accelerate the use of these models to elucidating disease mechanisms and testing candidate therapeutics.

TABLE 1
Summary of MORF Mouse Line Cell Type Specific Labeling Efficiencies

| | | | MORF + | | |
|---|---|---|---|---|---|
| | | | cells | Total cells | Labeling |
| Cell type (antibody) | Cre line | MORF line | counted | counted | efficiency |
| Striatal MSN (DARPP-32) | Drd1-Cre | MORF1 | 59 | 2436 | 4.8%* |
| Striatal MSN (DARPP-32) | Drd2-Cre | MORF1 | 52 | 2813 | 3.7%* |
| Striatal MSN (DARPP-32) | Rgs9-Cre | MORF1 | 116 | 4154 | 2.8% |
| Substantia Nigra | TH-Cre | TIGRE-MORF | 24 | 561 | 4.7% |
| Dopaminergic (TH) | | | | | |
| Striatal MSN (DARPP-32) | Drd1-Cre | MORF2 | 15 | 2149 | 1.4%* |
| Striatal MSN (DARPP-32) | Drd2-Cre | MORF2 | 38 | 3203 | 2.4%* |
| Striatal MSN (DARPP-32) | Rgs9-Cre | MORF2 | 83 | 3720 | 2.2% |
| Cortical Interneuron | Pvalb-Cre | MORF2 | 33 | 2120 | 1.6% |
| (Parvalbumin) | | | | | |
| Cerebellar Purkinje Cell | Pcp2-Cre | MORF2 | 19 | 1941 | 1.0% |
| (Calbindin) | | | | | |
| Retinal Horizontal Cell | Cx57-iCre | MORF2 | 68 | 3825 | 1.8% |
| (Calbindin) | | | | | |
| Striatal MSN (DARPP-32) | Drd1-Cre | MORF3 | 36 | 1389 | 5.2%* |
| Striatal MSN (DARPP-32) | Drd2-Cre | MORF3 | 39 | 1593 | 4.9%* |
| Cerebellar Purkinje Cell | Pcp2-Cre | MORF3 | 28 | 1786 | 1.6% |
| (Calbindin) | | | | | |
| Cortical Interneuron | Pvalb-Cre | MORF3 | 99 | 3352 | 2.9% |
| (Parvalbumin) | | | | | |
| Cortical Interneuron | SST-Cre | MORF3 | 75 | 1979 | 3.8% |
| (Somatostatin) | | | | | |
| Retinal Horizontal Cell | Cx57-iCre | MORF3 | 175 | 15254 | 1.2% |
| (P5; Calbindin) | | | | | |
| Cortical Microglia (Iba1) | Cx3cr1- | MORF3 | 141 | 4049 | 3.5% |
| | CreERT2 | | | | |
| | (100 mg/kg | | | | |
| | TAM; 5 days) | | | | |
| Cortical Astrocyte | Aldh111- | MORF3 | 148 | 4250 | 3.5% |
| (S100j3) | CreERT2 | | | | |
| | (100 mg/kg | | | | |
| | TAM; 3 days) | | | | |

*Drd1 and Drd2 cells make up −50% of DARPP-32+ MSNs in the Striatal respectively, therefore the labeling efficiency calculation was made using one half of the total cells counted as the denominator.

TABLE 2
Breeding results from TIGRE-MORF line crossed with different
Cre lines compared to expected Mendelian ratios.

| | | | | | | | | Wild | | |
|---|---|---|---|---|---|---|---|---|---|---|
| Cre line | Cell type | MORF+ | | Cre+ | | MORF+/Cre+ | | type | | p-value |
| Drd1-Cre | Direct Striatal MSN | 6 | (5) | 10 | (5) | 0 | (5) | 4 | (5) | 0.015* |
| Adora2a-Cre | Direct and indirect | 9 | (6.75) | 6 | (6.75) | 0 | (6.75) | 12 | (6.75) | 0.009** |
| | Striatal MSN | | | | | | | | | |
| Rbp4-Cre | Cortical layer V neurons | 12 | (6.25) | 3 | (6.26) | 0 | (6.25) | 10 | (6.25) | 0.001** |
| Camk2a- | Excitatory cortical | 19 | (14) | 14 | (14) | 7 | (14) | 16 | (14) | 0.134 |
| CreERT2 | neurons | | | | | | | | | |
| TH-Cre | Dopaminergic neurons | 3 | (4.75) | 7 | (4.75) | 5 | (4.75) | 4 | (4.75) | 0.606 |

Expected genotype abundance in parenthesis (χ² Test);
*p < 0;05,
**p < 0.01

TABLE 3
Total number of mice generated from MORF lines crossed with different
Cre lines to examine sparse Cre-dependent MORF-labeling.

| Cre line | MORF line | Mice imaged |
|---|---|---|
| Drd1-Cre | MORF1 | 2 |
| Drd2-Cre | MORF1 | 2 |
| Rgs9-Cre | MORF1 | 3 |
| TH-Cre | MORF1 | 2 |
| Ella-Cre | MORF1 | 2 |
| Pcp2-Cre | MORF1 | |
| Pvalb-Cre | MORF1 | |
| Nestin-Cre | MORF1 | |
| TH-Cre | TIGRE-MORF | 4 |
| Camk2-CreERT2 | TIGRE-MORF | 2 |
| A2a-Cre | MORF2 | |
| Drd1-Cre | MORF2 | 4 |
| Drd2-Cre | MORF2 | 8 |
| Pcp2-Cre | MORF2 | 3 |
| Pvalb-Cre | MORF2 | 3 |
| Rgs9-Cre | MORF2 | 4 |
| Cx57-iCre | MORF2 | 2 |
| Cx57-iCre (P5) | MORF2 | 2 |
| Cx57-iCre (P10) | MORF2 | 3 |
| Cx57-iCre (P15) | MORF2 | 3 |
| Kcng4-Cre | MORF2 | 4 |
| Rbp4-Cre | MORF3 | 8 |
| Drd1-Cre | MORF3 | 10 |
| Drd1-Cre (PO) | MORF3 | 2 |
| Drd2-Cre | MORF3 | 22 |
| Drd2-Cre (PO) | MORF3 | 2 |
| Cx57-iCre | MORF3 | 3 |
| Cx57-iCre (P5) | MORF3 | 7 |
| Cx57-iCre (P4) | MORF3 | 4 |
| Cx57-iCre (P10) | MORF3 | 2 |
| Pcp2-Cre | MORF3 | 2 |
| Pvalb-Cre | MORF3 | 6 |
| Pvalb-Cre (PO) | MORF3 | 2 |
| Pvalb-Cre (P17) | MORF3 | 2 |
| SST-Cre | MORF3 | 2 |
| Cx3cr1-CreERT2 | MORF3 | 7 |
| Aldh111-CreERT2 | MORF3 | 2 |
| Etv1-CreERT2 | MORF3 | 7 |
| Cux2-CreERT2 | MORF3 | 5 |
| Camk2-CreERT2 | MORF3 | 28 |
| VIP-Cre | MORF3 | 4 |
| Nestin-Cre | MORF3 | 2 |
| | Total | 184 |

### REFERENCES

Adalbert, R., and Coleman, M. P. (2013). Neuropathol Appl Neurobiol. 39, 90-108.
Araki, K., et al. (1997). Journal of biochemistry 122, 977-982.
Ascoli, G. A., et al. (2007). J. Neurosci. 27, 9247-9251.
Badea, T. C., et al. (2003). J Neurosci. 23, 2314-2322.
Badea, T. C., et al. (2009). PLoS ONE 4, e7859.
Baena, V., et al. (2019) Methods Cell Biol. 152, 41-67.
Bargmann, C. I., and Newsome, W. T. (2014). JAMA Neurol. 71, 675-676.
Bhargava, A., and Fuentes, F. F. (2010). Molecular Biotechnology 44, 250-266.
Biezonski, D. K., et al. (2015) J Comp Neurol. 523, 1175-89
Boije, H., et al. (2016). Front Neuroanat. 10, 77.
Buschiazzo, E., and Gemmell, N. J. (2006). Bioessays 28, 1040-1050.
Cajal, S. R. (1909). Histologie Du Systeme Nerveux de L'homme & Des Vertebres (ed. Maloine, A.) (Paris: Maloine, 1909). Translated by Swanson, N. & Swanson, L. W. (Oxford University Press, 1995).
Chan, K. Y., et al. (2017). Nat Neurosci. 20, 1172-1179.
Chapot, C. A., et al. (2017). J Physiol. 595, 5495-5506.
Chaya, T., et al. (2017). Sci Rep. 7, 5540.
Chung, W. S., et al. (2015). Nat. Neurosci. 18, 1539-1545.
Daigle, T. L., et al. (2018). Cell 174, 465-480.
De Biase, L. M., et al. (2017). Neuron 95, 341-356.
Dong, H. W. (2008). The Allen Reference Atlas: A Digital Color Brain Atlas of the C57BL/6J Male Mouse (Hoboken, New Jersey: Wiley).
Drinnenberg, A., et al. (2018) Neuron 99, 117-134.
Duman, R. S., and Aghajanian, G. K. (2012). Science 338, 68-72.
Dunn, F. A., and Wong, R. O. (2012). J Neurosci. 32, 10306-10317.
Ecker, J. R., et al. (2017). Neuron 96, 542-557.
Economo, M. N., et al. (2016). Elife 5, e10566.
Ero, C., et al. (2018). Front Neuroinform. 12, doi.org/10.3389/fninf.2018.00084.
Feng, L., et al. (2015) eNeuro. 2, ENEURO.0049-14.2014.
Fu, H., Hardy, J., and Duff, K. E. (2018). Nat Neurosci. 21, 1350-1358.
Gertler, T. S., et al. (2008). J Neurosci. 28, 10814-10824.
Gouwens, N. W., et al., (2019). Nat Neurosci. 22, 1182-1195.
Guérin, C. J., et al. (2019) Methods Cell Biol. 152, 87-101.
Hameyer, D., et al. (2007). Physiol Genomics 31, 32-41.
Haverkamp, S., and Wässle, H. (2000). J Comp Neurol. 424, 1-23.
Hayashi, S., and McMahon, A. P. (2002). Dev Biol. 244, 305-18.
Helmstaedter, M., et al. (2013). Nature 500, 168-74.
Heintzmann, R., and Ficz, G. (2013). Breaking the Resolution Limit in Light Microscopy. In Methods in Cell Biology, G. Sluder and D. E. Wolf, ed. (Amsterdam, Netherlands: Elsevier B.V.), pp. 525-544.
Herculano-Houzel, S. (2012). Proc Natl Acad Sci USA 109, 10661-10668.
Herculano-Houzel, S., et al. (2006). Proc. Natl. Acad. Sci. U.S.A. 103, 12138-12143.
Hintiryan, H., et al. (2016). Nat Neurosci. 19, 1100-1114.
Hirano, A. A., et al. (2016). eNeuro 3, https://doi.org/10.1523/ENEURO.0148-15.2016.
Hordeaux, J., et al. (2019). Mol Ther. 27, 912-921.
Huang, Z. J., and Paul, A. (2019). Nat Rev Neurosci. 20, 563-572.
Huckfeldt, R. M., et al. (2009). Nat Neurosci. 12, 35-43.
Ibrahim, L. A., et al. (2018). Cereb Cortex. bhy154, 1-14.
Jefferis, G. S., and Livet, J. (2012). Curr Opin Neurobiol. 22, 101-110.
Jeon, C. J., et al. (1998). J Neurosci. 18, 8936-8946.
Kasthuri, N., et al. (2015). Cell 162, 648-661.
Khakh, B. S., et al. (2017). Trends in neurosciences 40, 422-437.
Lakso, M., et al. (1996). Proc Natl Acad Sci USA 93, 5860-5865.
Langfelder, P., et al. (2008). Bioinformatics 24, 719-720.
Lee, C. Y. D., et al. (2018). Neuron 97, 1032-1048.
Li (2019)
Li, Y. C., et al. (2004). Mol Biol Evol. 21, 991-1007.
Lichtman, J. W., and Denk, W. (2011). Science 334, 618-623.
Livet, J., et al. (2007). Nature 450, 56-62.
Long, J. M. and Holtzman, D. M. (2019). Cell 179, 312-339.
Lu, X. H., and Yang, X. W. (2017). Sci Rep. 7, 43915.
Luo L. (2007). Brain Res Rev. 55, 220-227.
Luo, L., et al. (2008). Neuron 57, 634-660.
Madisen, L., et al. (2010). Nat Neurosci. 13, 133-140.
Markram, H., et al. (2015). Cell 163, 456-492.
Masland, R. H. (2012). Neuron 76, 266-280.
Maaten, L. V. D., and Hinton, G. (2008). J Mach Learn Res, 9, 2579-2605.
Menalled, L. B., et al. (2012). PLoS ONE 7, e49838.
Oh, S. W., et al. (2014). Nature 508, 207-214.
Parkhurst, C. N., et al. (2013). Cell 155, 1596-609.
Peichl, L., and Gonzalez-Soriano, J. (1994). Vis Neurosci. 11, 501-517.
Peng, H., et al. (2010). Nat Biotechnol. 28, 348-353.
Poché, R. A., and Reese, B. E. (2009). Development 136, 2141-2151.
Rapaport, D. H., et al. (2004). J Comp Neurol. 474, 304-324.
Raven, M. A., et al. (2007). J Neurosci. 27, 3540-3547.
Renier, N., et al. (2014). Cell 159, 896-910.
Renier, N., et al. (2016). Cell 165, 1789-1802.
Richardson, D. S., and Lichtman, J. W. (2015). Cell 162, 246-257.
Rosario, A. M., et al. (2016). Mol Ther Methods Clin Dev. 3, doi.org/10.1038/mtm.2016.26.
Shaner, N. C., et al. (2013). Nat Methods 10, 407-409.
Soriano, P. (1999) Nat Genet. 21, 70-71.
Soto, F., et al. (2018) Elife 7, e30388.
Srinivasan, R., et al. (2016). Neuron 92, 1181-1195.
Steinmetz, N. A., et al. (2017). eNeuro. 4, https://doi.org/10.1523/ENEURO.0207-17.2017.
Stepanyants, A., et al. (2004). Neuron 43, 251-259.
Ströh, S., et al. (2018). J. Neurosci. 38, 2015-2028.
Svitkina, T. (2009). Imaging cytoskeleton components by electron microscopy. In Methods Mol Biol., R. Gavin, ed. (New York: Humana Press), vol. 586, pp. 187-206.
Swanson, L. W., and Lichtman, J. W. (2016). Annu Rev Neurosci. 39, 197-216.
Taniguchi, H., et al. (2011). Neuron 71, 995-1013.
Tasic, B., et al. (2011). Proc Nati Acad Sci USA 108, 7902-7907.
Tremblay, R., et al. (2016). Neuron 91, 260-292.
von Bartheld, C. S., et al. (2016). J Comp Neurol. 524, 3865-3895.
Viswanathan, S., et al. (2015). Nat Methods 12, 568-576.
Wang, N., et al. (2014). Nat Med. 20, 536-541.
Wang, Y., et al. (2019). bioRxiv 675280, doi.org/10.1101/675280.
Whitney, I. E., et al. (2011). Proc Natl Acad Sci USA 108, 9697-9702.
Wilcox, R. R. (2011). Introduction to robust estimation and hypothesis testing. (Cambridge, Mass.: Academic Press).
Winnubst, J., et al. (2019). Cell 179, 268-281.
Young, R. W. (1985). Anat Rec. 212, 199-205.
Yoshimatsu, T., et al. (2014). Nat Commun. 5, doi.org/10.1038/ncomms4699.
Zeng, H., and Sanes, J. R. (2017). Nat Rev Neurosci. 18, 530-546.
Zingg, B., et al. (2014). Cell 156, 1096-1111.
Zong, H., et al. (2006). Cell 121, 479-492.

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A nucleic acid construct comprising, in operable linkage, a translation start site, an optional spacer, a polycytosine mononucleotide repeat, and an open reading frame (ORF), wherein the polycytosine repeat and the ORF are out of frame with respect to the translation start site.

2. The construct of claim 1, wherein the polycytosine mononucleotide repeat consists of between 5 and 50 cytosines.

3. The construct of claim 1, wherein the polycytosine mononucleotide repeat consists of 22 cytosines (C₂₂).

4. The construct of claim 1, wherein the spacer comprises at least 3 base pairs.

5. The construct of claim 1, wherein the spacer comprises between 3 and 100 base pairs.

6. The construct of claim 1, wherein the optional spacer sequence encodes one or two Myc tags.

7. The construct of claim 1, wherein the ORF encodes a fluorescent protein.

8. The construct of claim 7, wherein the fluorescent protein is GFP, RFP, tdTomato, and/or mNeonGreen.

9. The construct of claim 1, wherein the ORF encodes an immunoreporter.

10. The construct of claim 1, wherein the ORF encodes a polypeptide or protein that has enzymatic activities.

11. The construct of claim 9, wherein the immunoreporter comprises one or more epitope tags, optionally selected from the group consisting of: simian virus 5-derived epitope (V5), myelocytomatosis viral oncogene (Myc), hemagglutinin (HA) and/or a FLAG tag.

12. The construct of claim 1, wherein the ORF encodes a tandem fusion of two or more spaghetti monster immunoreporters.

13. The construct of claim 1, wherein the ORF encodes a tandem fusion of two or more spaghetti monster immunoreporters comprising 20 or more V5 epitope tags.

14. The construct of claim 1, wherein the ORF encodes a membrane insertion signal.

15. The construct of claim 1, wherein the ORF is fused with a farnesylation signal.

16. The construct of claim 15 wherein the farnesylation signal is a Ras CAAX domain.

17. The construct of claim 1, further comprising a polyadenylation signal downstream from the ORF.

18. The construct of claim 1, further comprising a protein coding sequence between the translation start site (ATG) and the polycytosine mononucleotide repeat.

19. The construct of claim 1, further comprising a protein coding sequence positioned after a translation start site and a polycytosine mononucleotide repeat.

20. The construct of claim 18 or 19, wherein the protein coding sequence is an uninterrupted protein coding sequence or a genomic DNA sequence comprising a mixture of exons and introns.

21. The construct of claim 1, which is shown in FIG. 1.

22. The construct of claim 1, further comprising a promoter, a transcriptional stop sequence, and two site-specific recombinase binding sites flanking the transcriptional stop sequence, wherein the promoter is upstream of the recombinase binding sites, and wherein each of the preceding elements is upstream of the translation start site.

23. The construct of claim 22, wherein the transcriptional stop sequence contains at least one polyadenylation signal.

24. The construct of claim 22, wherein the recombinase binding sites are LoxP sites, and wherein the LoxP sites are oriented such that Cre recombinase excises the transcriptional stop sequence.

25. The construct of claim 23, wherein the recombinase binding sites are Frt sites, and wherein the Frt sites are oriented such that Flp recombinase excises the transcriptional stop sequence.

26. The construct of claim 23, wherein the promoter is a cytomegalovirus early enhancer element and chicken beta actin (CAG) promoter.

27. A nucleic acid construct comprising, in operable linkage, a translation start site, a spacer, a polyguanine mononucleotide repeat, and an open reading frame (ORF), wherein the polyguanine mononucleotide repeat and the ORF are out of frame with respect to the translation start site.

28. The construct of claim 27, wherein the polyguanine mononucleotide repeat consists of between 5 and 50 guanines.

29. The construct of claim 27, wherein the polyguanine mononucleotide repeat consists of 22 guanines (G₂₂).

30. The construct of claim 27, wherein the spacer comprises at least 3 base pairs and up to about 100 base pairs.

31. The construct of claim 27, wherein the spacer sequence encodes one or two Myc tags.

32. The construct of claim 27, wherein the ORF encodes a fluorescent protein.

33. The construct of claim 32, wherein the fluorescent protein is GFP, RFP, tdTomato, and/or mNeonGreen.

34. The construct of claim 27, wherein the ORF encodes an immunoreporter.

35. The construct of claim 27, wherein the ORF encodes a polypeptide or protein that has enzymatic activities.

36. The construct of claim 34 wherein the immunoreporter comprises one or more epitope tags, optionally selected from the group consisting of: simian virus 5-derived epitope (V5), myelocytomatosis viral oncogene (Myc), hemagglutinin (HA) and/or a FLAG tag.

37. The construct of claim 27, wherein the ORF encodes a tandem fusion of two or more spaghetti monster immunoreporters.

38. The construct of claim 27, wherein the ORF encodes a tandem fusion of two or more spaghetti monster immunoreporters comprising 20 or more V5 epitope tags.

39. The construct of claim 27, wherein the ORF encodes a membrane insertion signal.

40. The construct of claim 27, wherein the ORF is fused with a farnesylation signal.

41. The construct of claim 40, wherein the farnesylation signal is a Ras CAAX domain.

42. The construct of claim 27, further comprising a polyadenylation signal downstream from the ORF.

43. The construct of claim 27, further comprising a protein coding sequence between the translation start site (ATG) and the polyguanine mononucleotide repeat.

44. The construct of claim 27, further comprising a protein coding sequence that is positioned after a translation start site and a polyguanine mononucleotide repeat.

45. The construct of claim 43 or 44, wherein the protein coding sequence is an uninterrupted protein coding sequence or a genomic DNA sequence comprising a mixture of exons and introns.

46. The construct of claim 27, further comprising a promoter, a transcriptional stop sequence, and two site-specific recombinase binding sites flanking the transcriptional stop sequence, wherein the promoter is upstream of the recombinase binding sites, and wherein each of the preceding elements is upstream of the translation start site.

47. The construct of claim 46, wherein the transcriptional stop sequence contains at least one polyadenylation signal.

48. The construct of claim 46, wherein the recombinase binding sites are LoxP sites, and wherein the LoxP sites are oriented such that Cre recombinase excises the transcriptional stop sequence.

49. The construct of claim 46, wherein the recombinase binding sites are Frt sites, and wherein the Frt sites are oriented such that Flp recombinase excises the transcriptional stop sequence.

50. The construct of claim 46, wherein the promoter is a cytomegalovirus early enhancer element and chicken beta actin (CAG) promoter.

51. A cell comprising the construct of any of the preceding claims.

52. A non-human vertebrate comprising the cell of claim 51.

53. The vertebrate of claim 52 which is a mammal.

54. The vertebrate of claim 52 which is a mouse.

55. A method of producing sparse and stochastic labeling of Cre-expressing cells in a host mammal, the method comprising generating a mammal that expresses the construct of any one of claims 1-50.

56. A method of producing sparse and stochastic labeling of cells expressing one or more site-specific recombinases in a host mammal, the method comprising generating a mammal that expresses the construct of any one of claims 1-50.

57. The method of claim 56, wherein the one or more site-specific recombinases are selected from the group consisting of Cre, Flp, FlpO, Vika, and Dre.

58. The method of any of claims 55, 55 or 56, wherein the labeling reveals the complete morphology of the cells.

59. The method of claim 58, wherein the cells comprise neurons and/or non-neuronal cells in the central nervous system, peripheral nervous system, and/or peripheral tissues.

60. The method of claim 59, wherein the non-neuronal cells include microglia, astrocytes, oligodendrocytes, myeloid cells, endothelial cells, and/or cells of the hematopoietic system (T cells, B cells, monocytes, macrophages, dendritic cells).
